(19) United States
(12) Patent Application Publication
(10) Pub. No.: US 2009/0105786 A1
(43) Pub. Date: Apr. 23, 2009

(54) METHOD AND DEVICE FOR STRENGTHENING SYNAPTIC CONNECTIONS

(75) Inventors: Eberhard Fetz, Seattle, WA (US); Andrew Jackson, Hampstead (GB); Jaideep Mavoori, Bellevue, WA (US)

Correspondence Address: KAREN S. CANADY;CANADY & LORTZ LLP, COMMERCE PLAZA, 11340 WEST OLYMPIC BLVD., SUITE 275, LOS ANGELES, CA, 90064, US

(73) Assignee: University of Washington, Seattle, WA (US)

(21) Appl. No.: 12/255,408

(22) Filed: Oct. 21, 2008

Related U.S. Application Data
(60) Provisional application No. 60/981,663, filed on Oct. 22, 2007.

(51) Int. Cl. A61N 1/36

(52) U.S. Cl. 607/48

(57) ABSTRACT

The invention provides a method and device for inducing a conditioned neural response in a subject. The method comprises detecting spike activity in a first neural site in the subject; and delivering a stimulus pulse to a second neural site in the subject. The stimulus pulse is delivered within the time window for synaptic strengthening following the detecting of a spike. These two steps, detection and stimulation, are repeated continuously, typically for a day or two. The conditioned neural response is induced when a pattern of neural activity evoked by stimulation at the first neural site emulates a pattern of neural activity evoked by stimulation at the second neural site. The conditioned neural response persists for an extended period of time.

This application claims benefit of U.S. provisional patent application No. 60/981,663, filed Oct. 22, 2007, the entire contents of which are incorporated by reference into this application.

### STATEMENT REGARDING FEDERALLY SPONSORED RESEARCH

The invention disclosed herein was made with Government support under Grant No. NS012542, awarded by the National Institute of Neurological Disorders and Stroke; and Grant No. N00014-01-1-0676, awarded by the Office of Naval Research. The government has certain rights in this invention.

### TECHNICAL FIELD OF THE INVENTION

This invention relates generally to inducing reorganization of neural structure and function via timing-dependent plasticity. The invention provides a device and method for strengthening synaptic connections by continuously stimulating a neural site each time activity is detected in another neural site.

### BACKGROUND OF THE INVENTION

Loss of motor function (paralysis) associated with nervous system injuries including stroke and spinal cord injury results from a disruption of the neural pathways by which intent in the brain is converted into movements of the muscles. Recovery through traditional rehabilitation techniques is often incomplete and as yet no method of regenerating injured neural projections has demonstrated clinical viability. Studies suggest that what recovery does occur results from a reorganization of the nervous system such that spared neural pathways take over the function of damaged areas (Nudo et al., 1996, Science 272:1791-1794). The potential for neural connections (synapses) to change is known as plasticity, and is an active topic of neuroscientific research. In particular, it has been suggested that changes in synaptic efficacy depend critically on the precise timing of pre- and post-synaptic neural activity.

There remains a need for methods and materials capable of inducing recovery via reorganization of neural pathways. The invention disclosed herein addresses these needs and others by providing a means of inducing plasticity via conditioned stimulation of the neural structures.

### SUMMARY OF THE INVENTION

The invention provides a method of inducing a conditioned change in neural connections in a subject. The method of the invention can be used to strengthen or weaken synaptic connections. The method comprises detecting spike activity in a first neural site in the subject and delivering a stimulus pulse to a second neural site in the subject, wherein the stimulus pulse is delivered within 100 milliseconds of the detecting of a spike. In some embodiments, the stimulus pulse is delivered within 50 milliseconds of the detection of a spike. These two steps, detection and stimulation, are repeated continuously, typically for at least 12 hours. In some embodiments, the steps are repeated continuously for at least 24 or 48 hours. The conditioned neural change is induced when a pattern of neural activity evoked by stimulation at the first neural site emulates a pattern of neural activity evoked by stimulation at the second neural site. The conditioned neural change persists for an extended period of time. In some embodiments, the change in neural connections has been shown to persist for at least one week.

Synaptic connections are strengthened when the stimulus pulse is delivered after the detection of a spike in the first site at a delay that exceeds the conduction time between sites. Synaptic connections are weakened when the stimulus pulse is delivered before the arrival at the second site of the spike detected at the first site. The conduction time between sites will vary with the distance between the sites and the type of neural fibers. For example, the conduction between two cortical sites or between a cortical site and a spinal site is typically one or two msec, whereas the conduction time for small corticospinal fibers will be longer. Thus, a method of strengthening synaptic connections involves delivering the stimulus pulse between 1 and 100 msec after detecting a spike at the first site. A method of weakening synaptic connections involves delivering the stimulus pulse between 0 and 1 msec after detection of a spike in the first site.

The strengthening of synaptic connections can be confirmed by detecting a pattern of neural activity evoked by stimulation at the first neural site that emulates a pattern of neural activity evoked by stimulation at the second site. This can be manifested by an observable change in the output produced by intracortical microstimulation, by a strengthening or increase in the correlation between neural activity at the two sites, or by enhanced functional recovery in the affected areas. Examples of functional recovery include, but are not limited to, improved motor control and/or function, restoration of lost speech or language comprehension, improved sensation, and improved memory and/or learning capabilities.

In a typical embodiment, the delivering of a stimulus pulse is conditioned exclusively on the detecting of a spike such that no stimulus pulse is delivered to the second neural site except for one stimulus pulse delivered at a designated delay (e.g., 50 or 100 milliseconds) of each spike detected in the first neural site: The designated delay can also have a lower limit, in some embodiments, such as, for example, 1, 2, 5, 10 or 20 milliseconds. The delay is selected to control timing of presynaptic activity (associated with the first neural site) relative to postsynaptic depolarization at the second neural site. Accordingly, the delay can be adjusted to account for conduction time between the first and second neural sites, or for time delays associated with the methods of detection or stimulation employed in a particular embodiment.

In some embodiments, the stimulation is applied subdurally and proximate to the second neural site, such as within the neocortex or within a subcortical or spinal site. One example is stimulation via intracortical microstimulation (ICMS). Alternatively, the stimulation can be applied epidurally and target a subdural neural site. In a typical embodiment, the intracortical stimulus pulse has an intensity of about 40 μA, and a duration of about 200 μsec. Stimulus pulses of 10 to 100 μA (or up to 10 mA) and 0.2 to 1 msec are contemplated by the invention. In some embodiments, the stimulus pulse is selected so as to be is of an intensity below the movement threshold for a train of pulses at 100/sec.

Similarly, the detecting of spike activity can be with subdural or epidural recording. Examples of epidural detection of spike activity include, but are not limited to, detecting high-frequency components [40-500 cycles/sec] of an electrocorticogram (ECoG). In addition, cortical spike activity may be indirectly detected by correlated muscle activity recorded through an electromyogram (EMG). In such an embodiment, the second neural site, to which a stimulus pulse is delivered, can be the spinal cord. Alternatively, the second neural site can be in the motor cortex or other cortical areas.

Likewise, those skilled in the art can appreciate various means by which the pattern of neural activity evoked by stimulation at the first neural site is determined. For example, in one embodiment, the pattern is determined by analyzing cortical activity or motor activity. Those skilled in the art further appreciate a variety of means for detecting changes in synaptic connections in addition to effects evoked from the recording site. In one embodiment, for example, the change is determined by analyzing correlation between cortical activities at the associated sites or by motor activity.

In some embodiments, the first and second neural sites are in the cortex. The cortex can be, for example, motor cortex, sensory cortex, frontal cortex, occipital cortex, temporal cortex or parietal cortex. In some embodiments, the first neural site is in the motor cortex and the second neural site is in the spinal cord. Other combinations of neural sites are also contemplated, including other cortical areas, corpus callosum, subcortical areas (e.g., thalamus, hypothalamus, limbic system, basal ganglia, amygdala, hippocampus), cerebellum, olfactory bulb and/or tract, and muscles.

Various combinations of the first (recording) and second (detecting) sites are contemplated. For example, the first and second neural sites can be in the spinal cord, or the first neural site can be in the motor cortex and the second neural site can be in the spinal cord. Alternatively, the first site can be in the spinal cord and the second in the cortex, such as motor cortex or sensory cortex. In some embodiments, the first site is muscle (recording via EMG) and the second site is spinal cord or motor cortex.

In addition, the invention provides a neural prosthesis. Typically, the prosthesis comprises means for detecting spike activity in a first neural site in a subject; means for delivering a stimulus pulse to a second site in the subject; and means for conditioning delivery of a stimulus pulse to the second site exclusively on spike activity in the first neural site. Accordingly, no stimulus pulse is delivered to the second site except for one stimulus pulse delivered within 50 milliseconds of each spike detected in the first neural site. The second site can be a neural site or it can be muscle. In an alternative embodiment, the invention provides a prosthesis and method that allow a subject to directly control a previously paralyzed limb. In this embodiment, the prosthesis provides an artificial connection between motor cortex and muscle. While spike activity in the first site (cortex) leads to delivery of a stimulus pulse to the second site (target muscle), the same conditioning scenario is not required.

The prosthesis further comprises a support structure to which each of the preceding elements is attached. One example of a support structure is an electronic circuit. In a typical embodiment, the stimulus pulse is delivered between 1 and 50 milliseconds after the detecting of spike activity, although the time window, or delay, for stimulus delivery involves the same considerations discussed above in connection with the method of the invention.

In a typical embodiment, the means for detecting spike activity and the means for delivering a stimulus pulse each comprises an electrode array. The electrodes can be designed for intracortical, intraspinal, subdural or epidural placement. Typically, the means for conditioning delivery of a stimulus pulse on spike activity at the first neural site comprises a microprocessor or application specific integrated circuit.

### BRIEF DESCRIPTION OF THE DRAWINGS

FIGS. 1.1A-C illustrate the conditioning protocol and experimental design. FIG. 1.1A is a schematic of the artificial connection. Action potentials detected in the signal recorded from the Nrec electrode triggered electrical stimuli delivered to the Nstim electrode after a predefined delay. FIG. 1.1B shows the experimental setup for testing output effects of ICMS on the right wrist. FIG. 1.1C sets for the experimental sequence of ICMS testing and conditioning with the Neurochip.

FIGS. 1.2A-E demonstrate reorganization of motor cortical output following conditioning. FIG. 1.2A shows flexion-extension and radial-ulnar components of average wrist torque responses to ICMS before conditioning. Dashed lines indicate torque trajectory during 200 ms following stimulation. Solid arrows indicate the mean torque. Axes represent ±0.02 Nm for Nrec and Ctrl effects, and ±0.08 Nm for the Nstim effect. FIG. 1.2B shows peri-stimulus averages of rectified EMG response in three wrist muscles to ICMS. Axis lengths are 250 ms and 0.4 mV. Dark bar indicates duration of ICMS train. FIGS. 1.2C-D present corresponding data following two days of conditioning with a connection between Nrec and Nstim. FIG. 1.2E provides angle and standard error of mean torque response to ICMS over 18 days. Shading indicates the conditioning period between days 7 and 9. ICMS comprised 13 biphasic pulses at 300 Hz, current: 30 μA (Nrec), 40 μA (Nstim) and 50 μA (Ctrl). Session 3 in Table 1. Average of 20 stimulus trains throughout.

FIGS. 1.3A-B summarize conditioning results for multiple sites. FIG. 1.3A shows angular separation of Nrec and Ctrl ICMS effects from Nstim effect before and after conditioning for 17 sessions. Control data (blue points) are clustered around the dashed line of identity indicating that conditioning stimulation did not affect the direction of mean torque elicited by ICMS. Nrec data (red points) fall below the line, indicating the direction of mean torque shifted towards the response elicited from the Nstim. Arrows, indicate the data in FIG. 1.2. FIG. 1.3B shows average angular separation of Nrec and Ctrl effects from Nstim effect. The shift towards the Nstim effect was still evident 1 day after conditioning. Bars indicate s.e.m., **P=0.0005, *P=0.001 (n=17, two-tailed paired t-test relative to pre-conditioning data).

FIG. 1.4 shows dependence of conditioning effects on delay between spikes and stimuli. Graph shows average angular shift of Nrec effects towards Nstim effects per day of conditioning for different spike-stimulus intervals at different sites. Solid line connects group means for each interval. Bars indicate s.e.m. Dashed line indicates 95th percentile for control electrodes obtained from the previous experiment.

FIG. 1.5 illustrates the suggested mechanism for the conditioning effect documented in FIG. 1.2. Pre-conditioning ICMS predominantly activates distinct descending projections from Nrec to FCR, from Nstim to ECR, and from Ctrl to FCU. Conditioning during unrestrained behavior induces a strengthening of horizontal connections between Nrec and Nstim. Post-conditioning ICMS now activates ECR via horizontal projections to Nstim, as well as FCR via the direct projection.

FIG. 1.6A is a photograph of the Neurochip circuit boards.

FIG. 1.6B is a photograph of Neurochip implant including circuits, battery and microwire electrodes.

FIGS. 1.7A-G provide representative data. FIG. 1.7A shows sample raw traces of spikes and stimulus artifacts recorded by the Neurochip from the Nrec electrode (40 sweeps overlaid). In this case a 5 ms delay was interposed between spike and stimulation. FIG. 1.7B shows the mean rate of stimulation across 24 hours of conditioning (same cell as FIG. 1.2). Red line plots 1-min averages and shading indicates the maximum and minimum 1 second rate over consecutive minutes. Cyclical pattern during the night is characteristic of alternating quiet and active (REM) sleep phases. Sample spike waveforms throughout the recording shown above. FIG. 1.7C is a histogram of stimulation rate over 1 second bins during day-time and night-time recording. Arrows indicate mean stimulation rate. FIG. 1.7D is a plot of the auto-correlation function for firing rate. This plot was calculated for the same cell but a different recording period with no conditioning stimulation, during which firing rate and EMG were stored over consecutive 100 ms bins. Half-width at half maximum is 500 ms. FIG. 1.7E is an inter-spike interval histogram for the same cell calculated for data recorded during the torque-tracking task. Absence of short (<1 ms) intervals and unimodal distribution are indicative of a single unit. FIG. 1.7F is a polar plot of firing rate during the torque-tracking task for the eight target directions. Arrow shows the preferred direction calculated as a vector sum. Axes lengths indicate 20 Hz. g Cross-correlation functions between cell firing rate and rectified EMG from two wrist muscles during free behavior.

FIG. 1.8A shows the magnitude of mean isometric torques before, during and after conditioning for the data shown in FIG. 1.2.

FIG. 1.8B shows the mean ratio of post- to pre-conditioning torque magnitude for all 17 datasets. Bars indicate s.e.m. None of the sites show a significant change (P≧0.1, two-tailed paired t-test).

FIG. 1.8C provides a typical example of the effect of stimulating current on direction and magnitude of ICMS-evoked torque. As stimulating current is increased above threshold, the effect increases in magnitude but the direction remains constant. (13 pulses at 300 Hz, average of 20 stimulus trains per intensity, axes length ±0.01 Nm).

FIGS. 1.9A-C provide additional examples of conditioning experiments. FIG. 1.9A: Monkey Y, session 2, axes lengths (Nm): ±0.01 (Nrec and Ctrl), ±0.02 (Nstim), ICMS currents (μA): 25 (Nrec and Ctrl), 30 (Nstim); FIG. 1.9B: Monkey Y, session 4, axes lengths (Nm): ±0.025 (Nrec and Nstim), ±0.05 (Ctrl), ICMS currents (μA): 40 (Nrec and Ctrl), 70 (Nstim); FIG. 1.9C: Monkey K, session 17, axes lengths (Nm): ±0.01, ICMS currents (μA): 50 (Nrec), 100 (Nstim and Ctrl). Average of approx. 20 stimulus trains throughout.

FIGS. 1.10A-C provide an example of an unstable conditioning effect. FIG. 1.10A: Angle and standard error of mean torque response to ICMS over 12 days. Shading indicates the conditioning period between days 2 and 4. The direction of the ICMS effect at Nrec is reversed from extension to flexion by conditioning, but gradually shifts back to the extension direction between days 10-12. FIG. 1.10B: Average torque trajectory on days 2, 4, 6, 8, 10 and 12. FIG. 1.10C: Magnitude and standard error of mean torque response to ICMS effect. This dataset (monkey Y, session 2) is the same as in FIG. 1.9A.

FIGS. 1.11A-B are cross-correlation histograms of the spiking activity of pairs of neurons recorded on the same and different electrodes. FIG. 1.11A: Histograms compiled with a bin-width of 100 ms. Correlated activity on this time scale is widespread throughout the motor cortex. FIG. 1.11B: Histograms compiled with a 0.1 ms bin-width. Synchrony with a precision of milliseconds is highest between neighboring neurons recorded on the same electrode. The absence of spike-pairs around zero time-lag results from failed discrimination due to waveform overlap. Histograms compiled from 10 min recording while monkey K reached for food rewards. Electrode separation: 1.1 mm.

FIGS. 2.1A-G illustrate construction of the moveable microwire array. FIG. 2.1A: Guide-tubes were first aligned on parallel tungsten rods and fixed with dental cement. Two rows of six tubes positioned on each side of a piece of card to produce a 6×2 array. FIG. 2.1B: Guide-tubes were splayed at one end and more cement was applied. FIG. 2.1C: Weighted microwires hung parallel along one side of the connector. Epoxy was applied to electrically insulate the contacts and fix the wires. FIG. 2.1D: The connector block was rotated and Silastic was applied to the wires for strain relief. FIG. 2.1E: Finished implant. The wires ran in loops from the connector block to the guide-tube array. The guide-tubes were filled with antibiotic ointment and sealed at both ends with Silastic. FIG. 2.1F: The implant as it was fixed to the skull during surgery. The microwires can be seen entering the brain through a craniotomy. FIG. 2.1G: Cross-section showing microwires penetrating the pia mater anterior to the central sulcus (CS) through a craniotomy and dural opening. The pia mater was bonded to the edge of the craniotomy with cyano-acrylate glue and the craniotomy was sealed with gelfoam and dental cement.

FIGS. 2.2A-C are sample neuronal recordings from primary motor cortex. FIG. 2.2A: 1-second recording obtained immediately after repositioning a microwire under ketamine sedation (day 0). Subsequent days are indexed relative to this, which corresponds to the 77th day after implantation of the array. FIG. 2.2B: Superimposed spike waveforms for recordings obtained on day 0 and six subsequent days while the monkey was awake. The larger waveform disappeared abruptly towards the end of day 5. FIG. 2.2C: ISI histogram compiled for spikes recorded during trained behavior on day 5.

FIGS. 2.3A-D illustrate long-term performance of moveable microwire array. FIG. 2.3A: Peak-to-peak amplitude of all single-units (light grey circles) and multi-units (dark grey circles) recorded over the life-time of the implant in monkey Y. Each point represents the amplitude of the cell on the first day that it was recorded. Tick-marks above the plot indicate occasions when the microwires were repositioned. The trend-line is based on least-squares fitting through all single- and multi-unit amplitudes. FIG. 2.3B: Comparable plot for monkey K. FIG. 2.3C: Histogram of SNR values for single-unit (light grey) and multi-unit (dark grey) waveforms. FIG. 2.3D: Scatter plot of SNR versus peak-to-peak amplitude for all cells.

FIGS. 2.4A-D describes stable and unstable recordings obtained during unrestrained behavior using implanted Neurochip circuitry. FIG. 2.4A: Sample waveforms for one cell over a 17-day period after positioning the microwire (on day 0). FIG. 2.4B: Mean firing rate over consecutive 1-minute intervals (dark line) through 18 hours of recording for this cell from day 16 to day 17. Also shown in grey are the maximum and minimum firing rates obtained during 100 ms intervals within each minute. The firing rate was lower while the monkey was asleep during the night but was otherwise stable. FIG. 2.4C: Recorded firing rate and waveforms for a different cell that showed steadily reduced amplitude between days 7 and 8. The apparent decline in recorded firing rate actually results from failure of the smaller waveforms to satisfy the discriminator parameters. FIG. 2.4D: Recorded firing rate and waveforms for a cell exhibiting an abrupt change on day 4.

FIGS. 2.5A-C provide an analysis of overall cell stability. FIG. 2.5A: Mean rate of cell loss per day for different intervals of time following movement of the wires. Bars indicate the standard deviation of loss rate based on a binomial distribution of loss events. FIG. 2.5B: Predicted percentage of day 1 cells retained on each subsequent day calculated from mean loss rates (dashed line). Also shown is the actual percentage of day 1 cells which were subsequently recorded (solid line). FIG. 2.5C: Mean change in signed (solid circles) and absolute (filled circles) peak-to-peak amplitude per day for different intervals of time following movement of the wires. Error bars indicate standard error of the means.

FIGS. 2.6A-B illustrate similarity between spike shapes for the same neuron on different days versus different neurons. FIG. 2.6A: Spike-shape similarity was quantified by the peak of the normalized cross-correlation function between average waveforms. A value of 1 indicates identical spike shapes, irrespective of absolute spike amplitudes. FIG. 2.6B: Histogram of all spike-shape similarities for the same neuron on different days (dark line), and between all pairs of different neurons in the entire dataset (grey line).

FIGS. 2.7A-B are photomicrographs showing post-mortem histology from Monkey Y. FIG. 2.7A: Cresyl-stained coronal sections showing gliosis surrounding electrode tracks running down the anterior bank of the central sulcus (CS) along the grey matter (GM) and white matter (WM) border. FIG. 2.7B: Increased magnification of the end of one electrode track located close to large layer V pyramidal cells.

FIG. 3 illustrates the cortically evoked responses as measured by isometric torque vectors about the wrist. Upper set shows torques in 3 directions: radial-ulnar (z axis), flexion-extension (x-axis) and pronation-supination (y axis); lower set shows same data plotted for last two dimensions.

FIGS. 4A-4C show brain-controlled functional electrical stimulation (FES) of muscle. (4A) Cortical cell activity is converted to FES during peripheral nerve block. (4B) Example of cell activity controlling FES of paralyzed wrist extensors. Extensor (red) and center (grey shading) wrist torque targets were randomly presented. Monkeys learned to modulate smoothed cell rate to control proportional muscle stimulation. FES was delivered to muscles, with current proportional to cell rate above a stimulation threshold (0.4 mA/pps×[cell rate−16 pps]; ≦10 mA). (4C) Histograms of cell rates driving FES to acquire targets. Shading indicates target hold period and horizontal line denotes baseline rate.

FIGS. 5A-5C show brain-controlled FES of multiple muscles to restore graded wrist torque in two directions. (A) The monkey acquired targets at five levels of flexion-extension torque using activity of a single cell to grade FES to both flexor and extensor muscles. Flexor FES was proportional to rate above a threshold; extensor FES was inversely proportional to cell rate below a second threshold. (B) Average torques produced to satisfy the five targets. With the stimulator off (shading), torques were less than 10% of magnitudes used to acquire the targets (blue and red lines), confirming the efficacy of nerve block. (C) Histograms of cell rate used to acquire five targets (colored boxes at left). Horizontal lines indicate FES thresholds for flexor (blue) and extensor (red) stimulation.

FIGS. 6A-6C show cell directional tuning is unrelated to FES control. (A) Responses of an untuned and strongly tuned cell (solid symbols in B & C). Histograms show cell activity while acquiring peripheral torque targets (shading) in flexion-extension (F-E) and radial-ulnar (R-U) planes during un-paralyzed tracking. Horizontal lines denote baseline cell rates, and radial plots summarize activity. Maximum target acquisition rates during brain control of cursor (B) and brain-controlled FES (C) vs. directional tuning strength (n=38). Performance controlling a cursor directly with cell activity was correlated with cell tuning (B; r2=0.33, p<0.001); subsequent brain-controlled FES performance was uncorrelated with tuning (C; r2=0.03, p=0.33).

FIG. 7 shows monkey L simultaneously modulated activity of two neurons, each controlling proportional stimulation of a different muscle group when above threshold. L acquired randomly presented flexor (blue), extensor (red) and center (grey) targets by using Cell 1 to stimulate a flexor muscle (FCU; 0.2 mA/pps×[cell rate-34 pps]) and Cell 2 to stimulate extensor muscles (ECU & ED4,5; 0.4 mA/pps×[cell rate−12 pps]).

FIGS. 8A-8B show the effect of nerve block on ability to generate wrist torques. (8A) Infusion of local anesthetic near the median, ulnar and radial nerves (arrows) eliminated wrist torques as the monkey controlled a cursor using cortical cell activity. (8B) Cell activity while acquiring high (top row) and low (bottom row) rate targets was unchanged during nerve block onset shown in A. Shading indicates target hold period and horizontal line denotes baseline cell rate.

FIG. 9 shows the number of targets acquired at the beginning of practice and during peak performance as brain cells control a cursor directly or control muscle FES (n=42 cells). Monkeys improved with practice for both conditions (**p<0.001); control during peak performance for each condition was not different (p=0.66). Error bars are standard deviation.

FIG. 10 shows that target errors gradually decrease with practice. For the same cell shown in FIG. 1B-C, the percentage of center targets during which the monkey activated the stimulator in error over two days of practice. Task difficulty was increased at each arrow, and was equal at the beginning of day 1 and 2. Note the absence of errors by the end of the second day of practice. Inset waveforms show overlaid cell action potentials during 30s of FES control on day 1 and 2.

FIGS. 11A-11C show the autonomous Neurochip system (11A) was used to convert cortical activity to FES of a paralyzed muscle (FCU). Monkey L modulated cell activity to trigger a 1 s train of 2.5 mA pulses at 50/s each time the cell rate exceeded 8 pps (11B). L rapidly acquired flexor (blue) torque targets and produced less than 5% of this torque (blue line) with the stimulator off (grey shading; 11C).

### DETAILED DESCRIPTION OF THE INVENTION

The invention is based on the discovery of a novel method of inducing neural plasticity to effect lasting reorganization of neural sites. The invention relates to using action potentials sensed in upstream neurons to trigger activation of downstream sites during the time-window for synaptic strengthening. The invention is based on a surprising effect of continuous time-dependent stimulation of a neocortical site that is correlated with detection of an action potential in another neocortical site. This discovery provides a neural prosthesis and method for rehabilitation of neural pathways lost or weakened due to injury or disease.

### DEFINITIONS

All scientific and technical terms used in this application have meanings commonly used in the art unless otherwise specified. As used in this application, the following words or phrases have the meanings specified.

As used herein, “spike activity” refers to action potentials detected by monitoring, directly or indirectly, the electrical activity of neurons in a subject. Spike activity can be detected by intracortical electrodes using single unit recording, by monitoring the subject's neural activity subdurally or epidurally, with electrodes recording the electrocorticogram (ECoG), or by monitoring peripheral or distal activity correlated with the targeted spike activity. For example, a subject's electromyogram (EMG) in a given muscle can be monitored to detect spike activity in a site of motor cortex that evokes movement in that muscle.

As used herein, a “stimulus pulse” means a pulse of electrical stimulation. A typical stimulus pulse is 10-100 μamperes delivered intracortically, or 0.2-5.0 milliamperes delivered subdurally or epidurally.

As used herein, “subject” refers to an animal, such as a mammal. Typically, the mammal is a primate, such as a human. Specific examples of “subjects” include but are not limited to: individuals requiring medical assistance; healthy individuals; individuals with limited function; and in particular, individuals with lost function due to traumatic injury or neurological disease.

As used herein, “a” or “an” means at least one, unless clearly indicated otherwise.

### Methods of the Invention

The invention provides a method of inducing changes in neural connections by strengthening synaptic connections in a subject. The method of strengthening synaptic connections comprises detecting spike activity in a first neural site in the subject; and delivering a stimulus pulse to a second neural site in the subject, wherein the stimulus pulse is conditioned or dependent on the detection of a spike in the first site. Typically, the stimulus pulse is delivered within 200 milliseconds of the detecting of a spike. In some embodiments, the stimulus pulse is delivered within 100 milliseconds, or within 50 milliseconds of the detection of a spike. The conditioning paradigm can employ one spike to one stimulus pulse, or multiple spikes and pulses, such as multiple spikes to one stimulus pulse, or one spike to multiple stimulus pulses. These two steps, detection and stimulation, are repeated continuously, typically for at least 12 hours. In some embodiments, the steps are repeated continuously for at least 24 or 48 hours, and longer periods of conditioning are feasible as well. The conditioning may be applied during waking or sleep, or both.

The change in physiological connections may be evident in a conditioned neural response whereby a pattern of neural activity evoked by stimulation at the first neural site emulates a pattern of neural activity evoked by stimulation at the second neural site. In some embodiments, the conditioned neural response or strengthening of synaptic connections is evidenced by an increase in the response evoked by stimulation at the first site. The conditioned neural response persists for an extended period of time, e.g., hours, days, weeks, months. In one example disclosed herein, the conditioned neural response has been shown to persist for at least one week.

In other embodiments, the method is used to weaken synaptic connections. Synaptic connections are strengthened when the stimulus pulse is delivered after the detection of a spike in the first site at a delay that exceeds the conduction time between sites. Synaptic connections are weakened when the stimulus pulse is delivered before the arrival at the second site of the spike detected at the first site. The conduction time between sites will vary with the distance between the sites and the type of neural fibers. For example, the conduction between two cortical sites or between a cortical site and a spinal site is typically one or two msec, whereas the conduction time for small corticospinal fibers will be longer. Thus, a method of strengthening synaptic connections involves delivering the stimulus pulse between 1 and 100 msec after detecting a spike at the first site. A method of weakening synaptic connections involves delivering the stimulus pulse between 0 and 1 msec after detection of a spike in the first site.

The strengthening of synaptic connections can be confirmed by detecting a pattern of neural activity evoked by stimulation at the first neural site that emulates a pattern of neural activity evoked by stimulation at the second site. This can be manifested by an observable change in the output produced by intracortical microstimulation, by a strengthening or increase in the correlation between neural activity at the two sites, or by enhanced functional recovery in the affected areas. Examples of functional recovery include, but are not limited to, improved motor control and/or function, restoration of lost speech or language comprehension, improved sensation, and improved memory and/or learning capabilities.

In a typical embodiment, the delivery of a stimulus pulse is conditioned exclusively on the detection of a spike such that no stimulus pulse is delivered to the second neural site except for one stimulus pulse delivered within the designated time window (200, 100 or 50 milliseconds) of each spike detected in the first neural site. The designated time window can also have a lower limit, such as, for example, 1, 2, 5, 10 or 20 milliseconds. To achieve strengthening of synaptic connections, the time window is selected to achieve concurrence of presynaptic activity (detected at the first neural site) with postsynaptic depolarization at the second neural site. Accordingly, the time window can be adjusted to account for differences in the distance between the first and second neural sites, or for time delays associated with the methods of detection and/or stimulation employed in a particular embodiment. To weaken synaptic connections, the time window is selected to deliver the stimulus pulse to the second site before the arrival at that second site of the spike detected at the first site.

In some embodiments, the stimulation is applied subdurally and proximate to the second neural site, such as within the neocortex or within a subcortical or spinal site. One example is stimulation via intracoritcal microstimulation (ICMS). Alternatively, the stimulation can be applied epidurally and target a subdural neural site. In a typical embodiment, the intracortical stimulus pulse has an intensity of about 40 μA, and a duration of about 200 μsec. Intracortical stimulus pulses of 10 to 100 μA and 0.2 to 1 msec are contemplated by the invention. In another embodiment, the subdural or epidural stimulus pulse has an intensity of about 1 mA, and a duration of about 200 μsec. Subdural or epidural stimulus pulses of 0.5 to 10 mA and 0.2 to 2 msec are contemplated by the invention. In some embodiments, the stimulus pulse is selected so as to be is of an intensity below the movement threshold for a train of pulses at 100/sec.

Similarly, the detecting of spike activity can be with subdural or epidural recording. Examples of epidural detection of spike activity include, but are not limited to, detecting high-frequency components [40-500 cycles/sec] of an electrocorticogram (ECoG). In addition, cortical spike activity may be indirectly detected by correlated muscle activity recorded through an electromyogram (EMG). In such an embodiment, the second neural site, to which a stimulus pulse is delivered, can be the spinal cord. Alternatively, the second neural site can be in the motor cortex or other cortical areas.

Likewise, those skilled in the art can appreciate various means by which the pattern of neural activity evoked by stimulation at the first neural site is determined. For example, in one embodiment, the pattern is determined by analyzing cortical activity or motor activity. Those skilled in the art further appreciate a variety of means for detecting changes in synaptic connections in addition to effects evoked from the recording site. In one embodiment, for example, the change is determined by analyzing correlation between cortical activities at the associated sites or by motor activity.

In some embodiments, the first and second neural sites are in the cortex. The cortex can be, for example, motor cortex, sensory cortex, frontal cortex, occipital cortex, temporal cortex or parietal cortex. In some embodiments, the first neural site is in the motor cortex and the second neural site is in the spinal cord. Other combinations of neural sites are also contemplated, including other cortical areas, corpus callosum, subcortical areas (e.g., thalamus, hypothalamus, limbic system, basal ganglia, amygdala, hippocampus), cerebellum, olfactory bulb and/or tract, and muscles.

Various combinations of the first (recording) and second (detecting) sites are contemplated. For example, the first and second neural sites can be in the spinal cord, or the first neural site can be in the motor cortex and the second neural site can be in the spinal cord. Alternatively, the first site can be in the spinal cord and the second in the cortex, such as motor cortex or sensory cortex. In some embodiments, the first site is muscle (recording via EMG) and the second site is spinal cord or motor cortex.

The methods of the invention can be used for rehabilitation to facilitate recovery of function due to neurological disease or damage. Subjects with a variety of conditions, as well as healthy individuals, can benefit from conditioning that strengthens synaptic connections between neural sites.

### Prosthetic Devices of the Invention

In addition, the invention provides a neural prosthesis. Typically, the prosthesis comprises means for detecting spike activity in a first neural site in a subject; means for delivering a stimulus pulse to a second neural site in the subject; and means for conditioning delivery of a stimulus pulse to the second neural site exclusively on spike activity in the first neural site. Accordingly, no stimulus pulse is delivered to the second neural site except for one stimulus pulse delivered within the designated time window of each spike detected in the first neural site. The second site can be a neural site or it can be muscle.

The prosthesis further comprises a support structure to which each of the preceding elements is attached. One example of a support structure is a circuit board. An exemplary embodiment of a prosthetic device of the invention is described in detail in Example 1 below. The device incorporates features of the Neurochip described in Mavoori et al., 2005, J Neurosci Methods 148:71-77; and Jackson et al., 2006, IEEE Trans Neural Sys Rehab Eng 14:187-190.

In a typical embodiment, the stimulus pulse is delivered between 1 and 50 milliseconds after the detecting of spike activity, although the time window for stimulus delivery involves the same considerations discussed above in connection with the method of the invention. In an alternative embodiment, the invention provides a prosthesis and method that allow a subject to directly control a previously paralyzed limb. In this embodiment, the prosthesis provides an artificial connection between motor cortex and muscle. While spike activity in the first site (cortex) leads to delivery of a stimulus pulse to the second site (target muscle), the same conditioning scenario is not required.

In a typical embodiment, the means for detecting spike activity and the means for delivering a stimulus pulse each comprises an electrode array. The electrodes can be designed for subdural or epidural placement. Typically, the means for conditioning delivery of a stimulus pulse on spike activity at the first neural site comprises a microprocessor. Example 2 below describes a compact, moveable microwire array that is suitable for long-term chronic unit recording in primate cerebral cortex. Those skilled in the art can appreciate other designs for electrode arrays suitable for subdural or epidural placement.

Those skilled in the art will also appreciate the ability to adapt devices known in the art for use with the method disclosed herein. Representative examples of other devices that involve detection and stimulation of neural sites include U.S. Patent Applications 20070032738, 20060173259, 20050240242, 20050119703, 20060009814, 20070032834, 20070179584, 200701123932, and 20060200206.

### EXAMPLES

The following examples are presented to illustrate the present invention and to assist one of ordinary skill in making and using the same. The examples are not intended in any way to otherwise limit the scope of the invention.

### Example 1

### Long-Term Reorganization of Motor Cortical Output Induced by an Electronic Neural Implant

This example demonstrates that the efficacy of neuronal connections is strengthened when there is a persistent causal relationship between pre- and postsynaptic activity. Such activity-dependent plasticity has been postulated to underlie the reorganization of cortical representations during learning, but direct in vivo evidence had previously been lacking. This example shows that stable reorganization of motor output can be induced by an artificial connection between two sites in the motor cortex of freely behaving primates. An autonomously operating electronic implant used action potentials recorded on one electrode to trigger electrical stimuli delivered at another location. Over one or more days of continuous operation, the output evoked from the recording site shifted to resemble the output from the corresponding stimulation site, consistent with potentiation of synaptic connections between the artificially synchronized populations of neurons. Changes persisted in some cases for over one week, while the output from sites not incorporated in the connection was unaffected. This novel method for inducing functional reorganization in vivo using physiologically-derived stimulus trains has practical application in neurorehabilitation following injury.

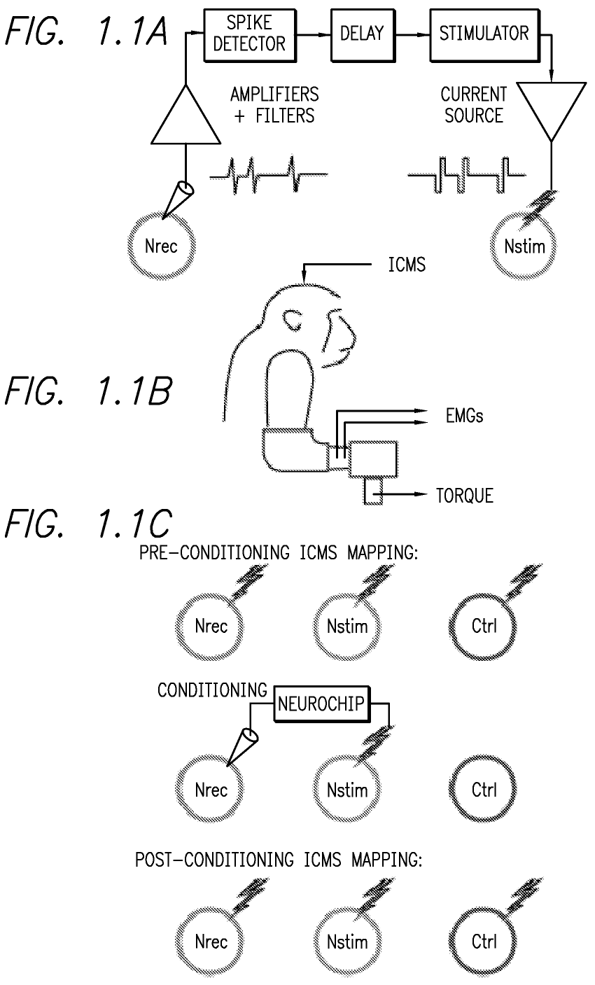

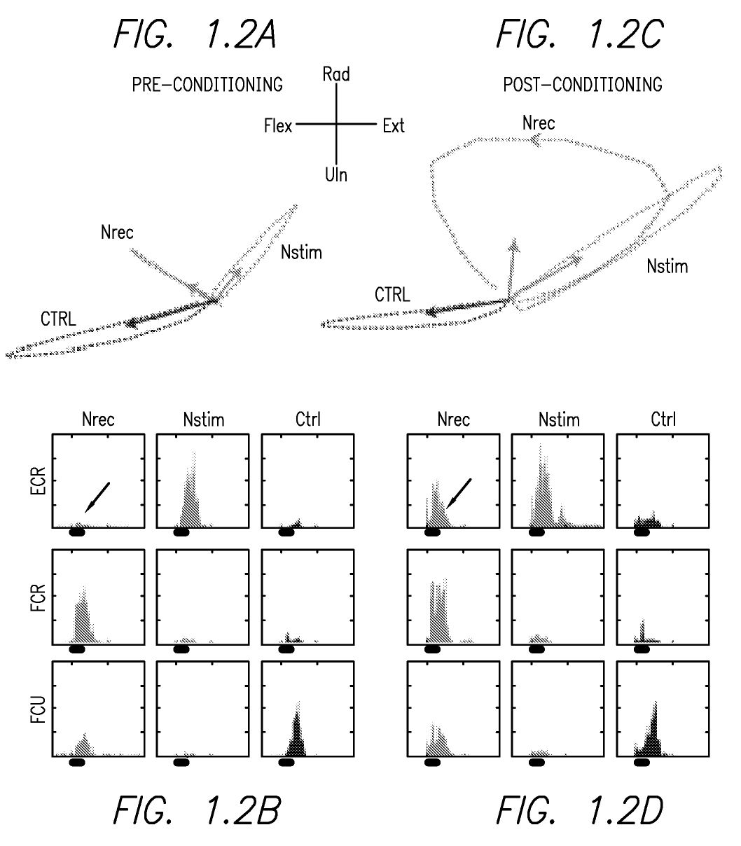

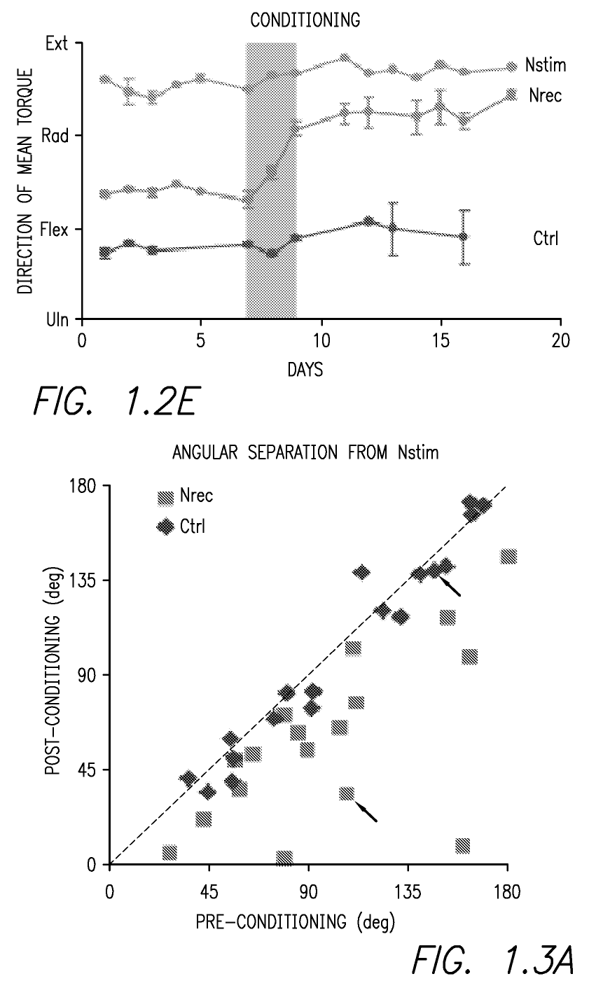

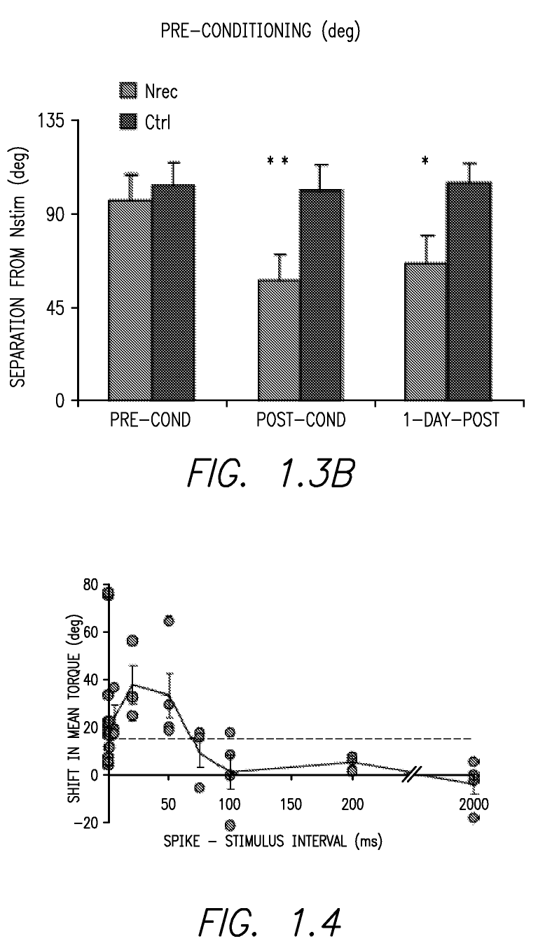

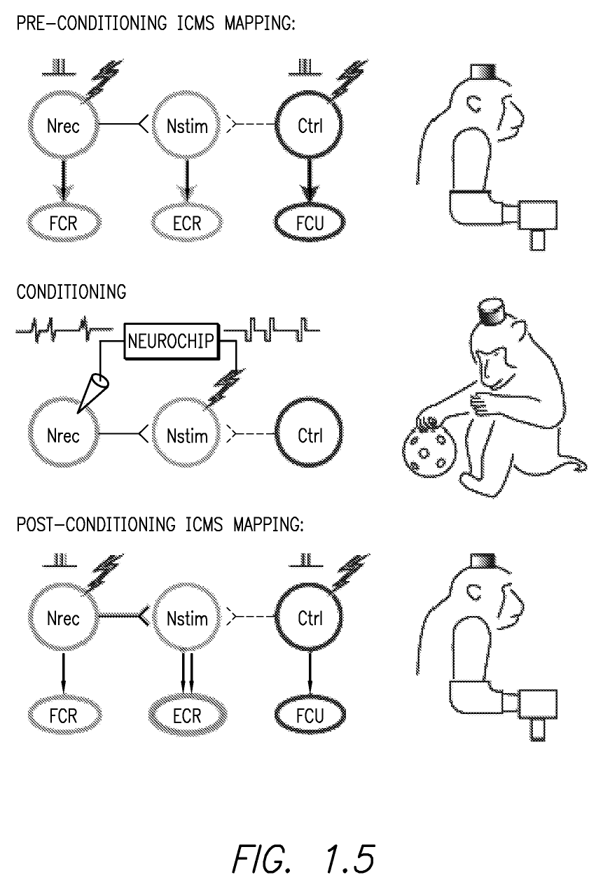

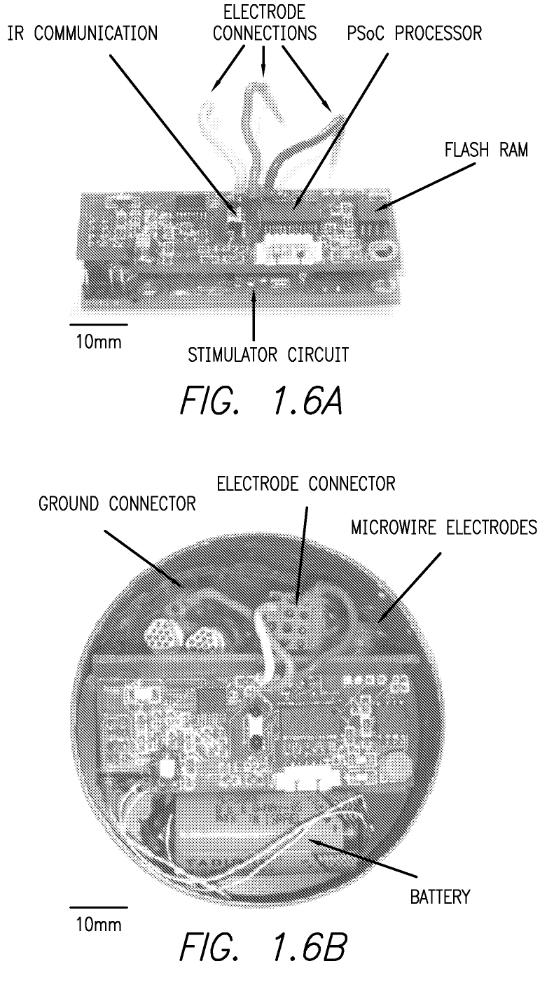

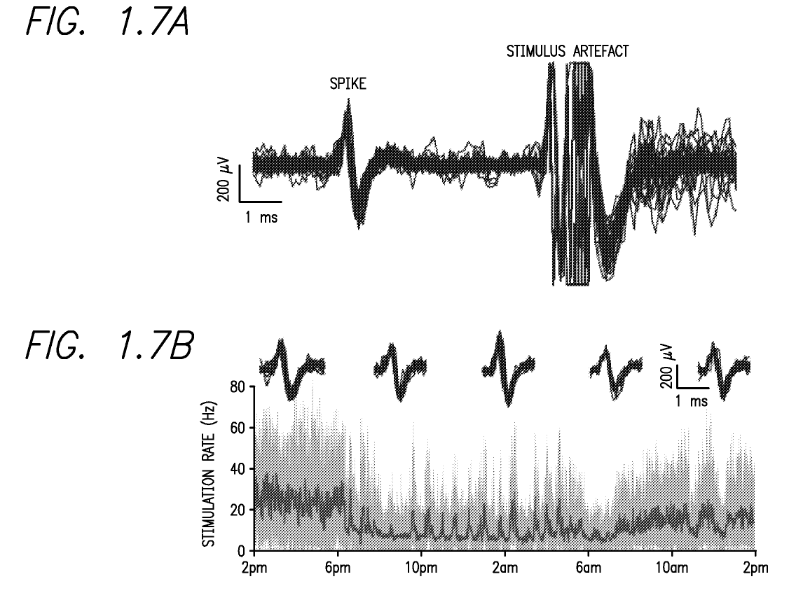

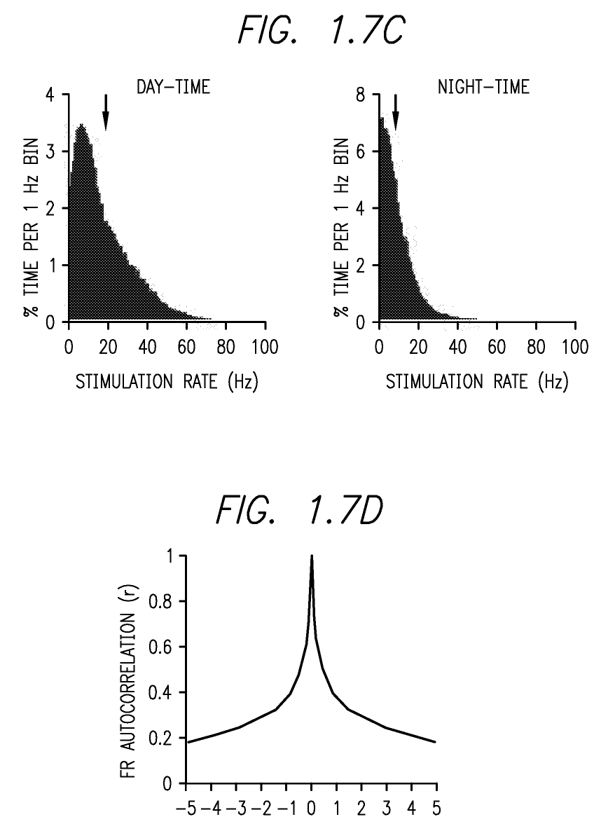

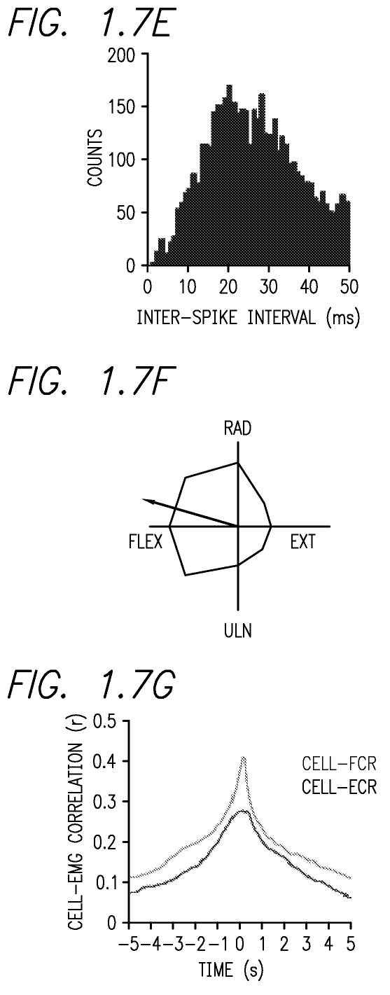

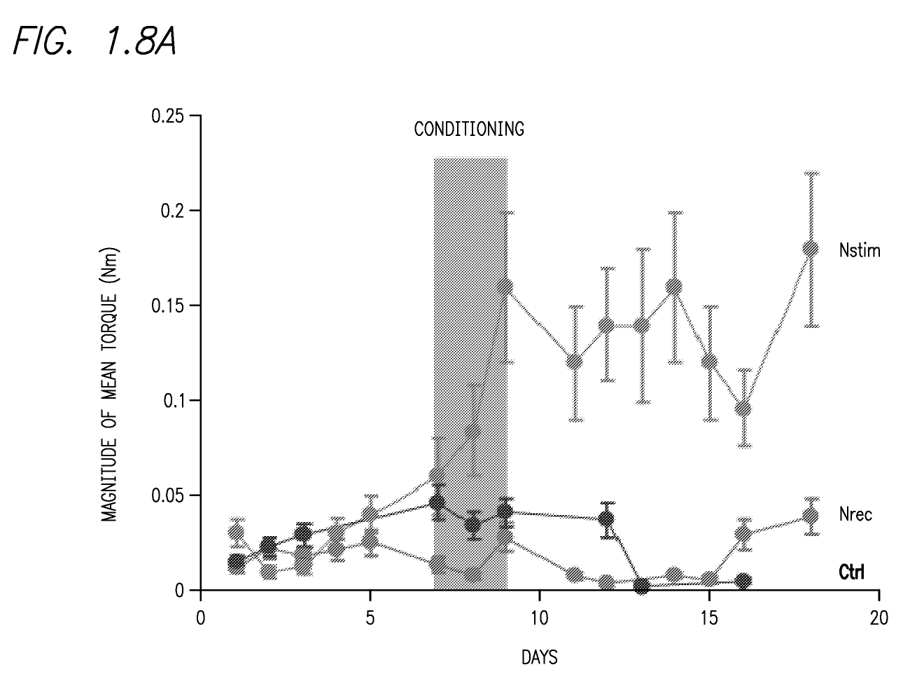

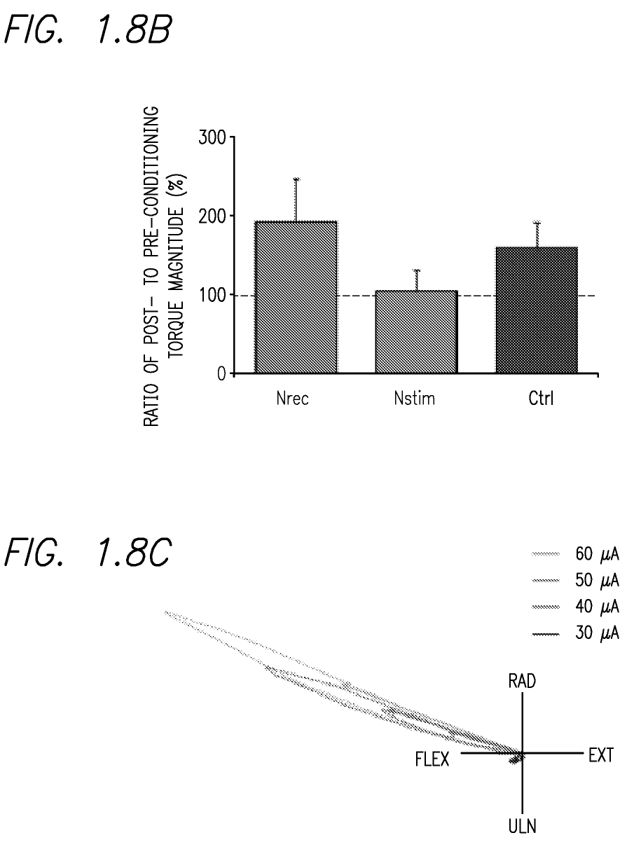

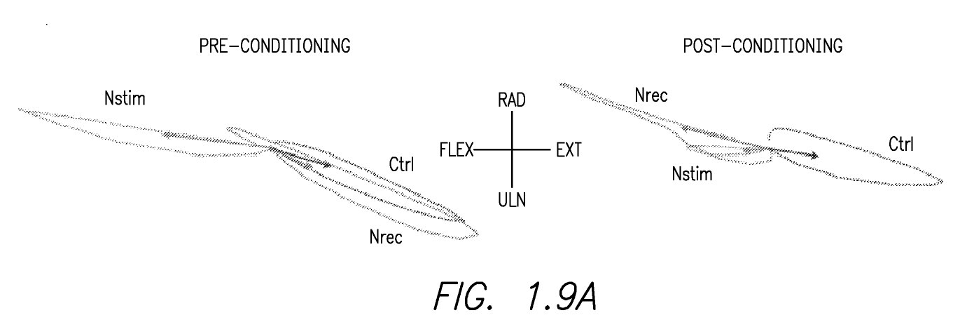

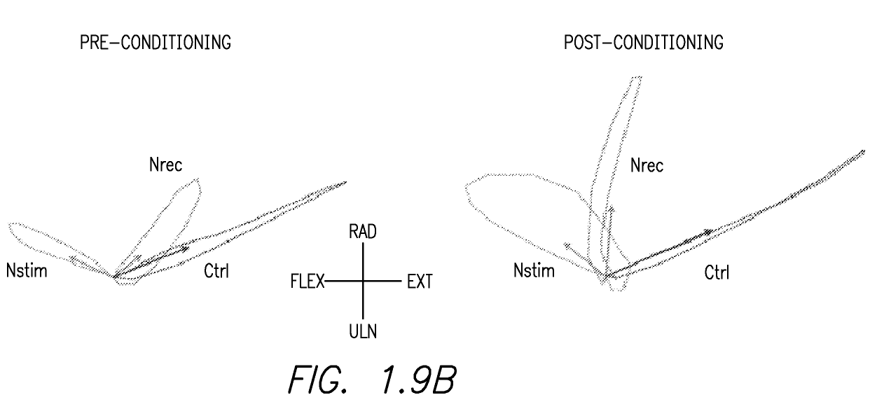

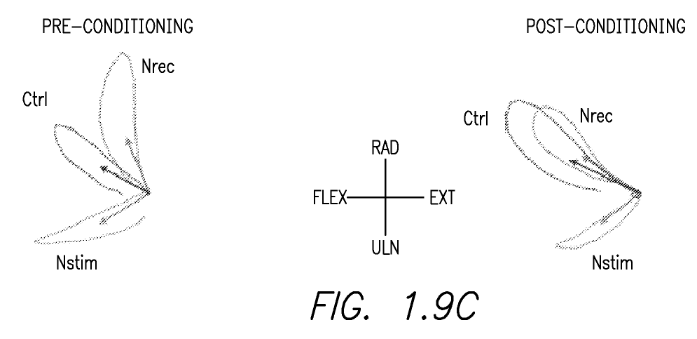

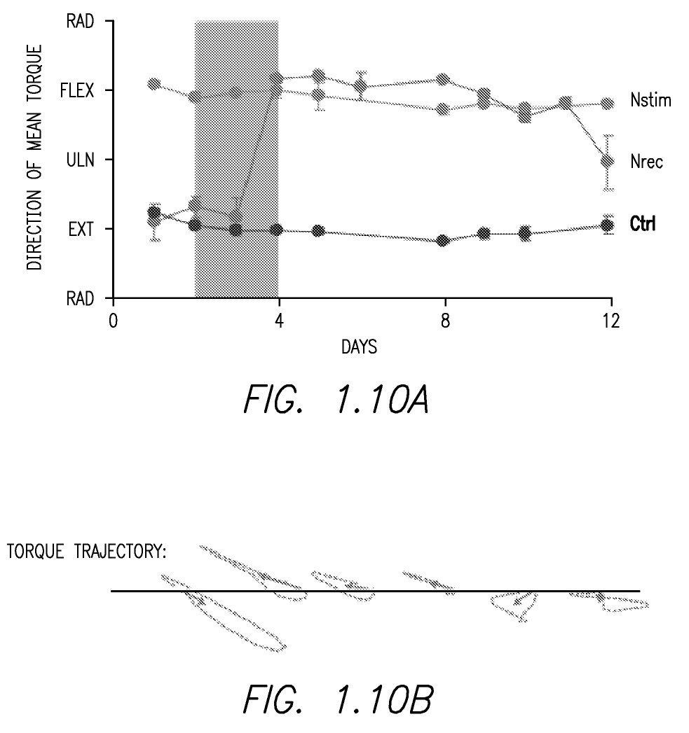

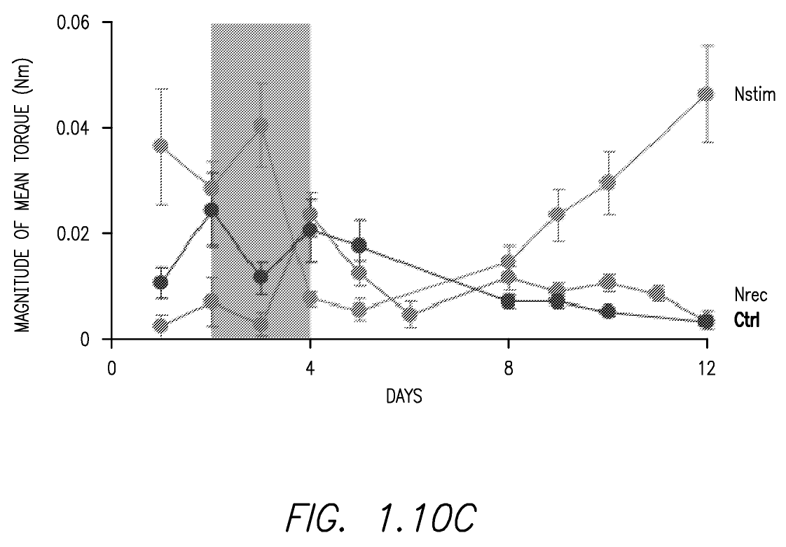

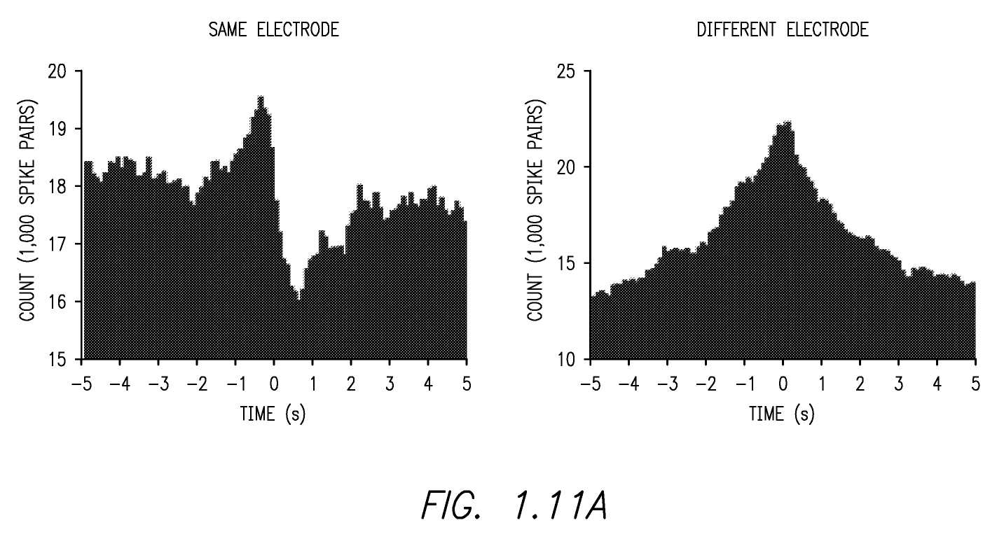

We are developing implantable electronic circuits (or Neurochips; FIG. 1.6) for neural recording and stimulation in freely behaving animals that could provide prosthetic connections to replace or augment damaged pathways in the nervous system¹⁸. [Superscript numerals appearing in this Example reference literature citations that can be found in Jackson A, et al. (2006) Nature, 444: 56-60.] A Neurochip creates an artificial connection between two sites by using action potentials recorded on one electrode to trigger electrical stimuli delivered to another (FIG. 1.1A). Once configured with appropriate recording and stimulation parameters, the Neurochip operates autonomously, allowing continuous spike-triggered stimulation over days of unrestrained behavior. Since the Neurochip creates a causal relationship between neural activities at connected sites, its long-term operation could also induce changes mediated by Hebbian mechanisms. Here we describe stable reorganization of movement representations in wrist area of primary motor cortex (M1) in monkeys resulting from artificial connections between pairs of electrodes in a chronically implanted array. We found that the motor output elicited from recording sites shifted towards the output evoked from stimulation sites. These changes occurred only when stimuli were delivered within 50 ms of recorded spikes, and the output evoked from neighboring control electrodes was unchanged. This demonstrates that natural patterns of cortical spiking in vivo during normal behavior can lead to input-specific, Hebbian plasticity when paired with appropriate stimulation. Plastic changes arising from such artificial connections could have clinical applications in rehabilitation following motor injury.

### Methods

Subjects. Experiments were performed using two male macacca nemestrina monkeys: Y (3 y/o, weight: 4.3 kg) and K (3 y/o, weight: 4.6 kg). All procedures were approved by the University of Washington Institutional Animal Care and Use Committee (IACUC).

Surgical procedure. The monkeys received pre- and post-operative corticosteroids (dexamethasone 1 mg/kg PO) to reduce cerebral edema. Surgery was performed under inhalation anesthesia (isoflurane 2-2.5% in 50:50 O2:N2O) and aseptic conditions. First, the scalp was resected and a craniotomy made over left M1 (A: 13 mm, L: 18 mm). The dura mater was removed and surface stimulation identified the precentral location from which movements of the wrist and hand could be evoked at the lowest threshold. The microwires were inserted through a 6×2 array of guide-tubes, pre-filled with antibiotic (Gentak, Akorn Inc.) and sealed with silastic (Kwik-Sil, WPI Inc.). The craniotomy was filled with gelfoam and sealed around the guide tubes with dental acrylic, leaving the wires free to be moved subsequently using forceps. Titanium skull-screws were used for reference grounding and to anchor a 6 cm diameter titanium chamber enclosing the microwires, electronics and battery. Surgery was followed by a full program of analgesics (buprenorphine 0.15 mg/kg IM and ketoprofen 5 mg/kg PO) and antibiotics (cephalexin 25 mg/kg PO).

Between each conditioning experiment, the monkeys were lightly sedated with ketamine (10 mg/kg IM) in order to clean the inside of the head casing (with dilute chlorohexadine solution followed by alcohol), and move the cortical microwires to sample new cells and ICMS effects. Post-mortem histology in monkey Y confirmed electrode tracks running down the grey matter in the bank of the central sulcus.

Neurochip electronics. For further description of the Neurochip circuitry see Mavoori et al., 2005, J Neurosci Methods 148:71-77. The electronics consisted of two 54 mm×22 mm printed circuit boards (PCBs) powered by one or two 2/3AA-sized 3.6 V lithium batteries (Tadiran Batteries Ltd.). The first PCB incorporated front-end amplification (1500×) and filtering (500 Hz-5 kHz), a Programmable System-on-Chip (PSoC, Cypress Semiconductor Co.), 8 Mb non-volatile memory and an infrared (IR) communication module. The second PCB incorporated a DC-DC converter producing a +14 V supply for the constant-current stimulator circuit. Dynamically-configurable modules within the PSoC performed additional amplification (1×-8×) and digitization (8-bit, 11.7 kHz) of the neural signal, and controlled the intensity and timing of stimulus pulses. The PSoC's 8-bit microprocessor core ran a dual time-amplitude window discriminator routine, operated the stimulator and handled IR communication with a laboratory computer. Custom software running in MatLab (Mathworks) was used to set recording, discrimination and stimulation parameters and to download data.

Cell tuning. For some cells we determined directional tuning from a center-out isometric torque-tracking task. A cursor provided visual feedback of flexion-extension and radial-ulnar torques and the monkey's task was to move the cursor from a central position to one of eight peripheral targets and hold for 1 second for a food reward. A one-factor ANOVA assessed the effect of direction on the number of spikes occurring during each 1-s hold period. For cells with a significant (P<0.05) directional tuning, a preferred direction vector was calculated by summing the torque direction vectors, each weighted by the mean firing rate during the hold period for that direction. The preferred direction of these cells is included in Table 1. Generally this was similar to the direction of torque effect elicited from the Nrec site. For some cells, we also recorded firing rate and EMG activity from two wrist muscles over 100 ms bins during different sessions of unrestrained behavior (with no conditioning) using the Neurochip system (Jackson et al, J. Neurophysiol 2007, 97: 360-374). These data were used to construct autocorrelation functions of firing rate (FIG. 1.7D), and cross-correlation functions between firing rate and muscle activity (FIG. 1.7G). As we have previously described (Jackson et al, J. Neurophysiol 2007, 97: 360-374.) cell firing rates often exhibited robust correlations with the activity of several muscles during free behavior.

Recording During conditioning, the Neurochip continuously calculated and stored the stimulation rate over consecutive 1-s bins. In addition, a 135 ms section of raw data was recorded every 8.8 minutes to verify the quality of recording and spike discrimination throughout. In this configuration, the 8 Mb of memory could hold over 24 hours of data. Discrimination windows remained constant during each day of conditioning. For conditioning sessions lasting longer than one day the battery was changed, data was downloaded and discrimination settings were updated once per day. Data recorded during the torque-tracking task were used to compile inter-spike interval histograms (FIG. 1.7E) to ensure the absence of short intervals which would indicate discrimination of more than one cell. However, during free behavior we were unable to store a sufficient amount of raw data to compile meaningful interval histograms. Therefore we cannot rule out the possibility of more than one cell intermittently appearing in the recording. In addition it is possible that slight changes in waveform shape could result in some spikes being undetected. Nevertheless, our experience of using this system is that overall firing rates are stable over periods of several days and waveforms extracted from the short sections of raw data remain constant over the recording period (FIG. 1.7B, Jackson et al, J. Neurophysiol 2007, 97: 360-374, and unpublished observations).

Conditioning stimulation. The intensity of Nstim conditioning stimulation was chosen such that spontaneous activity at Nrec caused occasional motor responses (which was typically at or slightly higher than the movement threshold for ICMS trains; see Table 1). For our initial experiments we chose to condition for several days to investigate the time-course of changes, but subsequently we used predominantly one day to maximize data collection. In many cases we were able to record cell activity on the Nstim electrode before and after conditioning indicating that our stimulation protocol did not damage the electrode or the tissue. As can be seen from FIG. 1.7A, stimulation produced an artifact on the recording electrode that lasted 2-3 ms, during which spikes could not be detected. For spike-stimulus delays of 0, 1 and 5 ms, spike detection was suspended from the time of the spike until 3 ms post-stimulation to avoid erroneous triggering from this artifact. This resulted in a maximum stimulation rate of 333, 250 and 125 Hz respectively; the rate over each 1-s bin typically did not exceed 100 Hz in any condition. However, for longer delays this method would have limited the maximum stimulation rate below typical cell firing rates, so an alternate approach was used to allow spike detection to continue during the delay and trigger the appropriate subsequent stimuli. A circular memory buffer continuously stored the spike train history as a spike count over consecutive intervals of time (bin-width: 1 ms for 50-200 ms delays, 8 ms for the 2000 ms delay). At the beginning of each interval, a stimulus was delivered if one or more spikes had occurred within the corresponding prior recording interval, and spike detection resumed after the stimulus artifact. In this way, the stimulus train could be delayed by up to 2 s relative to the spike train while retaining good temporal resolution and minimizing the number of undetected spikes.

Magnitude changes. In general we found that in the absence of conditioning stimulation, the direction of ICMS torques was more consistent than the magnitude of effects over consecutive days. As shown in FIG. 1.8C, the direction of torque typically did not change with stimulus intensity; higher currents simply produced larger effects in the same direction. For this reason, we used the same stimulation current to document ICMS effects before and after conditioning and analyzed primarily the effect of conditioning on the direction of torque responses. For completeness, FIG. 1.8B shows the ratio of the magnitudes of mean torque response before and after conditioning for the three sites averaged over all sessions. There was a 93% increase in the magnitude of response from Nrec, although this was not significant (P=0.1, two-tailed paired t-test). However, there was also a 60% increase in the magnitude of responses from control sites. These differences may reflect gradual changes at the electrode-tissue interface, for example a reduction of edema produced after moving the wires prior to each experimental session. There was only a 5% increase in magnitude of response from the Nstim site, and in some cases this response appeared to be suppressed following conditioning, but returned over the next few days (e.g. FIG. 1.10C). None of these magnitude differences achieved significance due to the high variability within the data, but it remains possible that repeated stimulation through the Nstim electrode over long periods may temporarily affect the electrode characteristics, reduce tissue excitability or increase local inhibition. This may also account for occasional changes in direction of torque response at Nstim sites (e.g. session 11). However, any long-term effects of Nstim stimulation or the tissue response to electrode positioning should have similar effects at both Nrec and Ctrl sites. Therefore this cannot explain the selective effect of conditioning on the direction of torque elicited from these electrodes, Which was restricted to the Nrec site.

Cross-correlation histograms. To document the coactivation patterns of cortical neurons, in some sessions we recorded spiking activity simultaneously from multiple microwires using a conventional instrumentation (MCP, Alpha-Omega) with the monkey seated in a chair. Spikes were discriminated using an off-line sorter (Plexon). Cross-correlation histograms were compiled from 10 minutes of recording while the monkey reached for food presented by the experimenter. Bin-widths of 100 ms and 0.1 ms were used. FIG. 1.11 shows example histograms for a cell pair recorded from the same electrode and a pair recorded from different electrodes with a representative separation (1.1 mm). Histograms compiled with the wide bin-width (FIG. 1.11A) revealed correlated firing rate modulations on the time-scale of behavior (several hundred milliseconds) over large distances within motor cortex as has been reported previously (Jackson et al, J. Neurophysiol 2007, 97: 360-374). The width of these cross-correlation peaks is comparable to the width of cell-muscle correlations observed during free behavior (FIG. 1.7G and ref. 19). The cell pair recorded from the same electrode also showed precise synchrony on a shorter time-scale of milliseconds (FIG. 1.11B; note that the absence of counts for bins around zero results from the failure to discriminate overlapping spike waveforms). However, such precise synchrony was not seen between the cell pair recorded from different electrodes. This is in agreement with a number of previous studies showing that precise synchrony between cell pairs decreases over several millimeters within primary motor cortex (refs. 25-27).

Neurochip implant. A full description of the Neurochip Brain-Computer Interface has been published previously (Mavoori et al., 2005, J Neurosci Methods 148:71-77). The battery-powered circuit allows continuous, long-term recording and stimulation during unrestrained behavior through an array of 12 chronically implanted, moveable tungsten microwire electrodes in M1 (diameter: 50 μm, impedance: 0.5 MD, interelectrode spacing: 500 μm). A microprocessor identified isolated action potentials from Nrec and instructed a stimulator circuit to deliver biphasic, constant-current pulses (0.2 ms/phase) to Nstim following a specified delay. Short sections of raw recording (sampled at 11.7 kHz) and stimulation rate in 1 second bins over the duration of conditioning were stored to on-board memory.

ICMS protocol. ICMS effects were documented using a current that was just above threshold for eliciting a torque response before conditioning and the same current was used throughout. The monkey sat in a chair with elbow and hand immobilized by padded restraints. A force transducer measured the 2D isometric torque produced at the wrist in the flexion-extension and radial-ulnar directions. During some sessions, EMG was recorded via pairs of stainless steel wires inserted trans-cutaneously into wrist muscles. Torque and EMG were recorded at 5 kHz. Off-line the torque trace was smoothed and down-sampled to 100 Hz. Trains of 13 biphasic ICMS pulses (0.2 ms/phase) at 300 Hz were delivered at 2 s intervals. Peri-stimulus averages of torque and rectified EMG profiles were compiled from 100 ms before to 500 ms after each stimulus. Traces in which the pre-stimulus torque level exceeded 0.02 Nm in any direction were excluded from the average. The trajectories in FIG. 1.2 connect the vector average of 2D torque across stimulus trains for consecutive sample points up to 200 ms post-stimulation. The vector average of this trajectory was used to determine the direction and magnitude of the mean torque response to ICMS at each site.

### Motor Cortex Plasticity Induced by an Artificial Connection

The output effects in the contralateral wrist evoked from cortical electrodes before, during and after conditioning with the artificial connection was tested with daily intracortical microstimulation (ICMS, FIG. 1.1B-C). FIG. 1.2A shows the preconditioning average trajectories of isometric wrist torque (dashed lines) for 200 ms following a train of stimuli delivered separately to each of three electrodes, designated Neurochip recording (Nrec), Neurochip stimulation (Nstim) and control (Ctrl). The mean torque, indicated by solid arrows, was toward the flexion direction for Nrec and Ctrl, and in the radial-extension direction for Nstim. FIG. 1.2B shows average rectified EMG responses to ICMS in three wrist muscles: extensor carpi radialis (ECR), flexor carpi radialis (FCR) and flexor carpi ulnaris (FCU).

The Neurochip was then programmed to deliver a single stimulus pulse to Nstim 5 ms after every action potential detected at Nrec (FIG. 1.7A). The cell at this site fired preferentially with flexion during torque-tracking and was correlated with both wrist flexor and extensor muscles during free behavior (FIG. 1.7F-G). The stimulus intensity (40 μA) was below movement threshold for a single pulse, but sufficient for occasional bursts of cell activity to elicit small muscle twitches while the monkey sat at rest. This connection produced no noticeable disruption of the control of active movements, probably because the effect of stimulation was weak compared to neural activity generating volitional movement. The artificial connection operated continuously for two days while the monkey moved unrestrained about the home cage. During this period, the Neurochip also recorded stimulation rate over consecutive 1 s bins (FIG. 1.7B). The mean rate of stimulation was 19 Hz during day-time behavior and 9 Hz during the night. This pattern was consistent with our observations of robust correlations between the firing rate of M1 neurons and muscle activity during natural behavior, and cycles of activity and quiescence during sleep19.

After two days of conditioning the mean torque generated by ICMS at Nrec had shifted to, the radial direction, towards the output effect produced from Nstim (FIG. 1.2C). The torque now followed a curved trajectory aligned initially with the Nstim trajectory, before returning to the flexion direction. This curved path was produced by a new pattern of muscle responses (FIG. 1.2b), which included initial activation of the extensor muscle ECR (black arrows), previously elicited only by ICMS at Nstim. The output from the control electrode was unaffected, indicating that the plastic change was caused by the pairing of neural activity at the Nrec site with stimulation of Nstim. In this case, the response from the Nstim site had also increased slightly (FIG. 1.8), but this was not generally the case. FIG. 1.2E plots the angle of mean torque produced by ICMS at each site for the days before, during, and after conditioning. The changes at the Nrec site developed gradually over the two days of conditioning and subsequently remained stable for one week.

### Summary of Conditioning Sessions

We investigated the effect of creating artificial connections between 17 different pairs of electrodes over separate conditioning sessions in two monkeys (Y: 8 sessions, K: 9 sessions). Conditioning lasted 1-4 days (mean 1.6 days) with Nstim currents in the range 25-80 μA (mean 48 μA) and delays of 0, 1 and 5 ms interposed between spike and stimulus (Table 1). ICMS effects were quantified by the angle of mean wrist torque relative to the pre-conditioning direction of Nstim effect. Control electrodes were chosen to have similar ICMS effects as the Nrec pre-conditioning. FIG. 1.3A summarizes the direction of mean torque produced by ICMS before and after each session. Points lying on the dashed line of identity represent ICMS effects that were unaffected by conditioning. Points lying below the line represent effects that moved towards the output effect of Nstim. Conditioning had no significant effect on the separation of Ctrl site effects from the Nstim direction, which changed on average by 2.1° (s.e.m. 2.3°, two-tailed paired t-test P=0.4). In contrast, Nrec effects moved towards the Nstim direction by an average of 38° (s.e.m. 9°, P=0.0005). In 13 of 17 individual sessions, Nrec effects rotated by an angle greater than the 95th percentile of the Ctrl distribution (15°). The mean (±s.e.m.) angular shift per day of conditioning was comparable for both animals (Y: 24°±80, K: 25°±7°). FIG. 1.3B shows average angular separations of Nrec and Ctrl effects from the Nstim direction before, just after, and 1 day after the end of the conditioning period. FIG. 1.9 shows example torque trajectories from these experiments. FIG. 1.10 shows the one session in which the change at the Nstim site gradually wore off, 6-8 days after the end of conditioning.

TABLE 1
Summary of pre- and post-conditioning ICMS effects.

| | | Nstim | | | | | | | | | | | |
|---|---|---|---|---|---|---|---|---|---|---|---|---|---|
| | | electrode: | | ICMS (pre-cond) | | | ICMS (post-cond) | | | Nrec electrode: | | | |
| | | | Intensity | Direction | | Magnitude | Direction | | Magnitude | | Cell PD | | Intensity |
| Session | Mky | MW | (μA) | (°) | | (Nm) | (°) | | (Nm) | MW | (°) | | (μA) |
| 1 | Y | 1 | 20 | −96 | (F) | 0.068 | −106 | (F) | 0.015 | 11 | 62 | (ER) | 40 |
| 2 | Y | 1 | 30 | −81 | (F) | 0.056 | −95 | (F) | 0.008 | 10 | 66 | (ER) | 25 |
| 3 | Y | 5 | 40 | 40 | (ER) | 0.072 | 60 | (ER) | 0.17 | 6 | −73 | (FR) | 30 |
| 4 | Y | 3 | 70 | −60 | (FR) | 0.041 | −45 | (FR) | 0.049 | 10 | 80 | (E) | 40 |
| 5 | Y | 12 | 50 | −69 | (FR) | 0.037 | −63 | (FR) | 0.037 | 10 | 103 | (EU) | 30 |
| 6 | Y | 9 | 30 | −66 | (FR) | 0.046 | * | | 0.002 | 11 | 109 | (EU) | 30 |
| 7 | Y | 9 | 30 | −63 | (FR) | 0.021 | * | | 0.001 | 11 | 86 | (E) | 30 |
| 8 | Y | 2 | 60 | 57 | (ER) | 0.014 | 58 | (ER) | 0.027 | 11 | −99 | (F) | 100 |
| 9 | K | 5 | 30 | −37 | (FR) | 0.0087 | −11 | (FR) | 0.019 | 10 | None | | 100 |
| 10 | K | 5 | 60 | 1 | (R) | 0.0093 | * | | 0.0011 | 10 | —^{†} | | 80 |
| 11 | K | 11 | 60 | 102 | (EU) | 0.0034 | −44 | (FR) | 0.011 | 12 | — | | 100 |
| 12 | K | 10 | 40 | −58 | (FR) | 0.027 | * | | 0.0006 | 9 | — | | 40 |
| 13 | K | 5 | 50 | −26 | (FR) | 0.0044 | −28 | (FR) | 0.013 | 10 | — | | 80 |
| 14 | K | 2 | 70 | −51 | (FR) | 0.0082 | * | | 0.0015 | 9 | — | | 150 |
| 15 | K | 11 | 30 | −67 | (FR) | 0.013 | −61 | (FR) | 0.028 | 5 | — | | 150 |
| 16 | K | 8 | 80 | −37 | (FR) | 0.014 | −57 | (FR) | 0.0033 | 10 | −106 | (FU) | 90 |
| 17 | K | 10 | 80 | −128 | (FU) | 0.012 | −134 | (FU) | 0.011 | 11 | 79 | (ER) | 50 |
| mean | | | 49 | | | 0.027 | | | 0.023 | | | | 69 |
| s.e.m. | | | 5 | | | 0.005 | | | 0.009 | | | | 10 |

| | | |
|---|---|---|
| ICMS (pre−cond) | ICMS (post−cond) | Conditioning |

| | | | | | | | | |
|---|---|---|---|---|---|---|---|---|
| Direction | Magnitude | Direction | Magnitude | Length | Delay | Intensity | ΔSep | ΔSep |
| (°) | (Nm) | (°) | (Nm) | (days) | (ms) | (μA) | (°) | (°/day) |

| | | | | | | | | | | |
|---|---|---|---|---|---|---|---|---|---|---|
| 66 | (ER) | 0.026 | 3 | (R) | 0.034 | 4 | 0 | 40 | 63 | 16 |
| 120 | (EU) | 0.011 | −73 | (F) | 0.022 | 2 | 0 | 30 | 151 | 76 |
| −66 | (FR) | 0.015 | 6 | (R) | 0.028 | 2 | 5 | 40 | 72 | 36 |
| 43 | (ER) | 0.031 | 5 | (R) | 0.074 | 2 | 5 | 40 | 38 | 19 |
| 111 | (EU) | 0.021 | 78 | (ER) | 0.064 | 2 | 5 | 60 | 33 | 17 |
| 18 | (ER) | 0.049 | −4 | (R) | 0.064 | 1 | 0 | 30 | 22 | 22 |
| 15 | (ER) | 0.062 | 8 | (R) | 0.043 | 1 | 0 | 50 | 7 | 7 |
| −52 | (FR) | 0.010 | −46 | (FR) | 0.098 | 2 | 0 | 60 | 6 | 3 |
| 41 | (ER) | 0.050 | −35 | (FR) | 0.011 | 1 | 0 | 30 | 76 | 76 |
| −62 | (FR) | 0.022 | −51 | (FR) | 0.0076 | 1 | 1 | 30 | 11 | 11 |
| −106 | (FU) | 0.039 | −16 | (R) | 0.055 | 2 | 1 | 60 | 34 | 17 |
| −33 | (FR) | 0.017 | −53 | (FR) | 0.0068 | 1 | 0 | 50 | 20 | 20 |
| −83 | (F) | 0.011 | −61 | (FR) | 0.013 | 1 | 1 | 50 | 22 | 22 |
| −10 | (R) | 0.046 | −30 | (FR) | 0.031 | 1 | 0 | 70 | 20 | 20 |
| 21 | (ER) | 0.016 | −13 | (R) | 0.071 | 2 | 0 | 30 | 34 | 17 |
| −92 | (F) | 0.010 | −87 | (F) | 0.011 | 1 | 0 | 80 | 5 | 5 |
| −18 | (R) | 0.014 | −51 | (FR) | 0.010 | 1 | 0 | 80 | 33 | 33 |
| | | 0.026 | | | 0.038 | 1.6 | | 48 | 38 | 25 |
| | | 0.004 | | | 0.007 | 0.2 | | 4 | 9 | 5 |

| | Ctrl electrode: | | ICMS (pre−cond) | | | ICMS (post−cond) | | | Conditioning | | | |
|---|---|---|---|---|---|---|---|---|---|---|---|---|
| | | Intensity | Direction | | Magnitude | Direction | | Magnitude | Nstim dir | | ΔSep | ΔSep |
| Session | MW | (μA) | (°) | | (Nm) | (°) | | (Nm) | (°) | | (°) | (°/day) |
| 1 | 5 | 100 | 101 | (E) | 0.025 | 91 | (E) | 0.026 | −96 | (F) | −10 | −3 |
| 1 | 10 | 80 | 100 | (E) | 0.011 | 103 | (E) | 0.019 | −96 | (F) | −3 | −1 |
| 2 | 2 | 25 | 110 | (E) | 0.016 | 90 | (E) | 0.020 | −81 | (F) | −2 | −1 |
| 3 | 1 | 50 | −107 | (F) | 0.046 | −100 | | 0.041 | 40 | (ER) | 7 | 7 |
| 4 | 5 | 40 | 63 | (ER) | 0.14 | 61 | | 0.21 | −60 | (FR) | 2 | 1 |
| 4 | 9 | 50 | 72 | (ER) | 0.077 | 59 | | 0.12 | −60 | (FR) | 13 | 7 |
| 5 | 5 | 40 | 71 | (ER) | 0.012 | 69 | | 0.018 | −69 | (FR) | 2 | 1 |
| 5 | 11 | 20 | 21 | (ER) | 0.086 | 12 | | 0.067 | −69 | (FR) | 9 | 9 |
| 6 | 10 | 20 | 24 | (ER) | 0.016 | 8 | | 0.026 | −66 | (FR) | 16 | 16 |
| 7 | 10 | 20 | 16 | (ER) | 0.035 | 18 | | 0.027 | −63 | (FR) | −2 | −2 |
| 8 | 9 | 70 | −95 | (F) | 0.023 | −85 | | 0.030 | 57 | (ER) | 10 | 5 |
| 9 | 7 | 60 | −80 | (F) | 0.011 | −71 | | 0.012 | −37 | (FR) | 9 | 9 |
| 11 | 9 | 100 | −11 | (R) | 0.0071 | −37 | | 0.046 | 102 | (EU) | −26 | −13 |
| 13 | 9 | 70 | −79 | (FR) | 0.028 | −85 | | 0.038 | −26 | (FR) | −6 | −6 |
| 16 | 4 | 60 | −71 | (FR) | 0.024 | −77 | | 0.018 | −37 | (FR) | −6 | −6 |
| 16 | 10 | 90 | −92 | (F) | 0.010 | −87 | | 0.011 | −37 | (FR) | 5 | 5 |
| 17 | 8 | 100 | −55 | (FR) | 0.012 | −59 | | 0.022 | −128 | (FU) | 4 | 4 |
| 17 | 4 | 80 | −74 | (FR) | 0.0082 | −89 | | 0.016 | −128 | (FU) | 15 | 15 |
| mean | | 60 | | | 0.033 | | | 0.043 | | | 2.1 | 2.6 |
| s.e.m. | | 7 | | | 0.008 | | | 0.011 | | | 2.3 | 1.7 |

All directions are given as angles measured clockwise from the radial direction. Parenthesized letters indicate Extension, Flexion, Radial and Ulnar directions. MW indicates the microwire number. Cell PD indicates the preferred direction of the cell at the Nrec site as determined by the torque-tracking task.
ΔSep gives the change in angular separation from the pre-conditioning direction of Nstim effect and Ctrl.
*indicates an ICMS effect which was suppressed after conditioning.
^{†}Directional tuning not available for these cells.

### Dependence of Plastic Changes on Stimulus Delay

The absence of conditioning effects at control sites suggests that the timing of stimulation relative to cell activity played a critical role in inducing plasticity. Neurons distributed widely throughout the motor cortex exhibit correlated firing on the time-scale of movements (typically several hundred milliseconds (Jackson et al, J. Neurophysiol 2007, 97: 360374); FIGS. 1.7G and 1.11A). Therefore the site specificity of our results indicates that a more precise coincidence between spikes and stimulation was required to induce plastic changes. We tested this hypothesis in a further series of experiments with monkey K by introducing longer delays of 20-2000 ms between the spike and stimulation pulse. FIG. 1.4 summarizes this data by plotting the angular shift of the Nrec effect towards the Nstim direction per day of conditioning as a function of stimulus delay. Significant shifts (P<0.05, two-tailed t-test) were obtained for intervals up to 50 ms, suggesting that a coincidence of spike and stimulus within this window is required for inducing plasticity. The average angular shift for delays of 20 and 50 ms (35°±5°) was slightly greater than had been obtained using the shorter delays (24°±5°), but this difference was not significant (P=0.25, two-tailed unpaired t-test).

### Discussion

These results may be explained by potentiation of horizontal pathways within the motor cortex10 such that post-conditioning ICMS delivered to Nrec activates additional muscle groups via Nstim (FIG. 1.5). Alternatively, plasticity could occur at other cortical or subcortical targets of converging projections from Nrec and Nstim sites. Repetitive high-frequency stimulation has been shown to expand movement representations in the motor cortex of rats12, 13, but a general expansion of local Nstim effects cannot account for the changes we saw at Nrec sites for several reasons. First and foremost, outputs from neighboring control electrodes were unaffected by conditioning. There was no significant difference in either the mean (±s.e.m.) distance from the Nstim site (Nrec: 1.09±0.16 mm, Ctrl: 0.95±0.12 mm, two-tailed unpaired t-test P=0.5) or ICMS currents used (Nrec: 68±10 μA, Ctrl: 60±7 μA, P=0.5). Furthermore, the angular shift of Nrec ICMS effects was not correlated with distance from the Nstim site (Pearson's r=−0.0007). Finally, no shifts occurred when stimuli were delayed more than 50 ms relative to the Nrec spikes, although the temporal pattern of stimulation was equivalent. These observations all indicate that the relative timing of Nrec spikes and Nstim stimulation was the critical factor for inducing plasticity.

Cellular studies of STDP have shown that plasticity at individual synapses is triggered by Ca2+ influx requiring both pre-synaptic glutamate release and post-synaptic depolarization to release the Mg2+ block of NMDA channels¹⁶. We propose that in our experiments, depolarization of local or down-stream neurons by Nstim stimulation induced potentiation of synapses concurrently activated by spikes arriving from the Nrec site. Inputs from other sites (i.e. Ctrl electrodes) were not potentiated since the timing of this pre-synaptic activity had no consistent correlation with post-synaptic depolarization. Previous studies have shown that synaptic inputs activated after post-synaptic depolarization can be depressed^{15, 16} so although some fraction of spikes from control sites would have arrived during the window for synaptic potentiation, these could be cancelled by a comparable number of inputs arriving during the window for synaptic depression. This would also explain why delays of around 20 ms tended to produce the strongest conditioning effects. With shorter delays more Nrec spikes would have arrived after stimulation, during the window for synaptic depression.

Associative plasticity has been demonstrated previously using paired stimulation of two input pathways in the cerebellum²¹, hippocampus²² and motor cortex²³. In contrast to these studies, we induced a functional reorganization using in vivo spike activity at one site to trigger stimulation of a second site. This constitutes a relatively direct test of Hebb's postulate, demonstrating that natural patterns of neuronal firing can lead to input-specific plasticity when paired with appropriate post-synaptic depolarization during normal behavior. It seems unlikely that the magnitude of this reorganization can be accounted for by altered projections only from the recorded neuron, since ICMS delivered to Nrec presumably activated populations of cells via local circuitry and temporal summation24. However, neighboring M1 neurons with similar output projections exhibit the maximal degree of synchronous discharge²⁵⁻²⁷ (FIG. 1.11B), so during conditioning many spikes from this population will be temporally correlated within the coincidence window for synaptic potentiation.

Our method for inducing plasticity shares similarities with in vivo cellular conditioning protocols in sensory areas that pair spontaneous or evoked neuronal activity with appropriate sensory stimulation²⁸⁻³⁰. However, the changes seen at the cellular level in those studies typically lasted for a few hours at most and were reversed by normal activity. The continuous conditioning over long periods of natural behavior implemented by our Neurochip system may account for the strength and stability of the effects described here, consistent with the finding that multiple sessions are required to induce stable LTP in rats in vivo11. Furthermore, conditioning was associated with volitional movements rather than externally imposed activation and continued during natural sleep, including rapid eye movement (REM) phases when motor cortical neurons can be highly active¹⁹. Sleep has been implicated in the consolidation of motor memory³¹, but the relative contribution of waking and sleeping periods to our results remains to be determined.

Artificial recurrent connections could provide a neural prosthesis to replace damaged pathways in the nervous system following injury¹⁸. Our results suggest that an additional rehabilitative consequence in cases of partial injury may be the strengthening of surviving projections between sites connected by the prosthesis. Functional reorganization is thought to be important in recovery from numerous movement disorders^{6,7} and novel stimulation protocols are being developed to aid this process³²⁻³⁴. Stimulation in real-time triggered from neural recordings during volitional movements could provide an effective means to selectively strengthen specific neural pathways during rehabilitation.

### Example 2

### Compact Moveable Microwire Array for Long-Term Chronic Unit Recording in Cerebral Cortex of Primates

This example describes a small, chronically implantable microwire array for obtaining long-term unit recordings from the cortex of unrestrained primates. It is presently optimized for use with non-human primates, but can be adapted for use with human subjects. After implantation, the depth of microwires can be individually adjusted to maintain large-amplitude action potential recordings from single neurons over many months. Data presented here were recorded from the primary motor cortex of two monkeys by autonomous on-board electronic circuitry. Waveforms of individual neurons remained stable for recording periods of several weeks during unrestrained behavior. Signal-to-noise ratios, waveform stability and rates of cell loss indicate that this method is particularly suited to uses relating to the neural correlates of processes extending over multiple days, for example learning and plasticity.

To obtain stable recordings of the same isolated single units over many days and to sample new cells with good signal-to-noise over long experimental periods we have developed a technique to implant moveable microwire arrays in the cortex of primates. The arrays are easily constructed from readily available materials, and yield high-quality, stable recordings of the same single units for a week or more at a time. By moving the wires periodically we have been able to maintain large spike waveforms and clean recordings over experimental periods of six months to a year from the same area of cortex. In this example we describe the construction of the implant and document the recording quality and stability of spike waveforms obtained from implants in the hand area of primary motor cortex (M1) of two monkeys.

### Methods

Implant design. The microwire implant consisted of 12 teflon-insulated 50 μm-diameter tungsten wires running inside polyamide guide-tubes. The wires entered the brain through an opening in the dura within a small craniotomy that was subsequently closed with dental cement. The guide-tubes themselves were filled with antibiotic ointment and sealed at both ends with Silastic. This waterproof seal prevented infection tracking into the brain and allowed experiments to be performed over many months with minimal chamber maintenance. The microwires slid freely through the Silastic seal, allowing the depth of each to be individually adjusted at any stage by grasping the wire above the guide-tube with forceps. The entire assembly was housed within a capped titanium casing attached with screws to the skull. For these experiments a 6 cm-diameter cylindrical casing contained a single microwire array in M1, as well as our Neurochip electronics and battery.

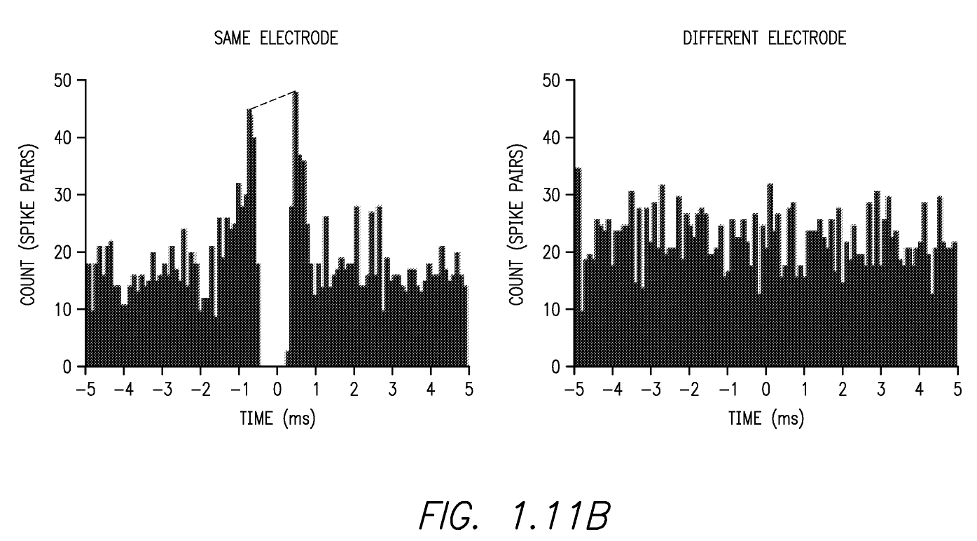

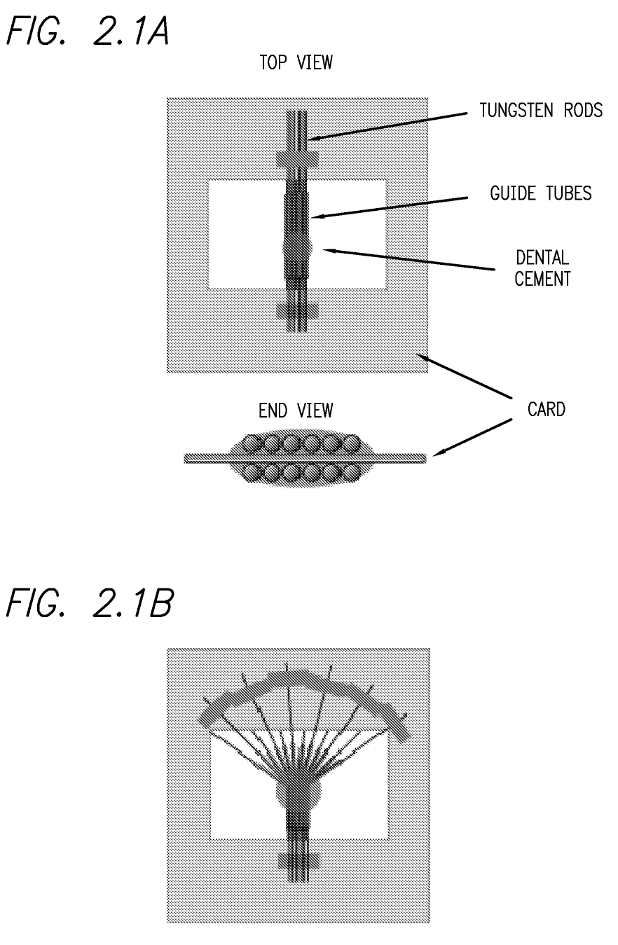

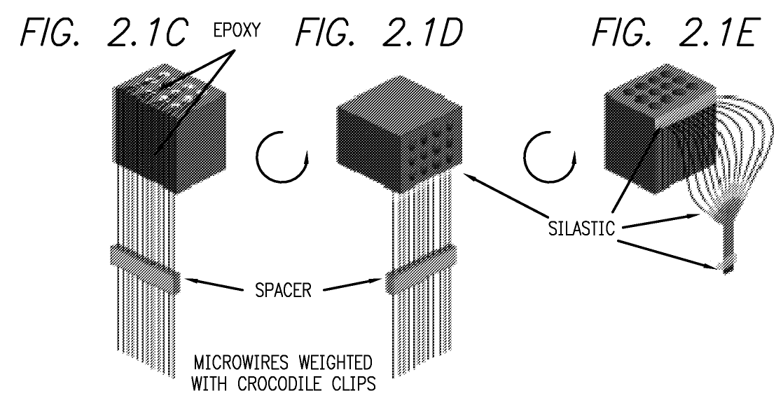

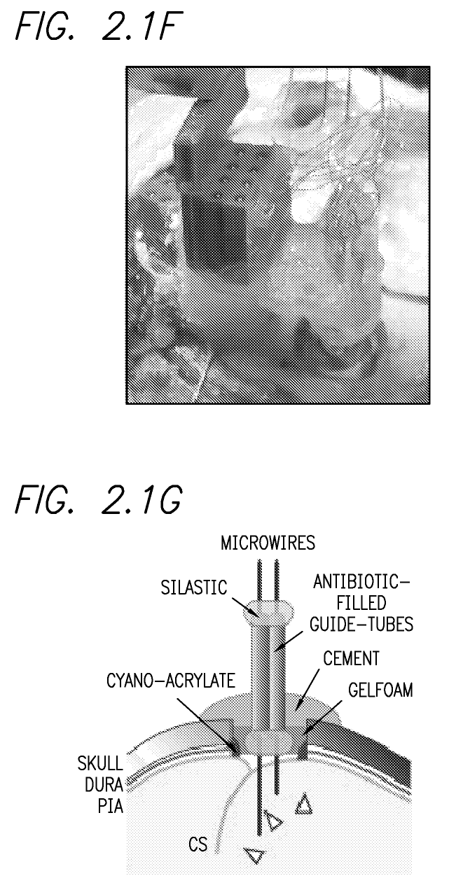

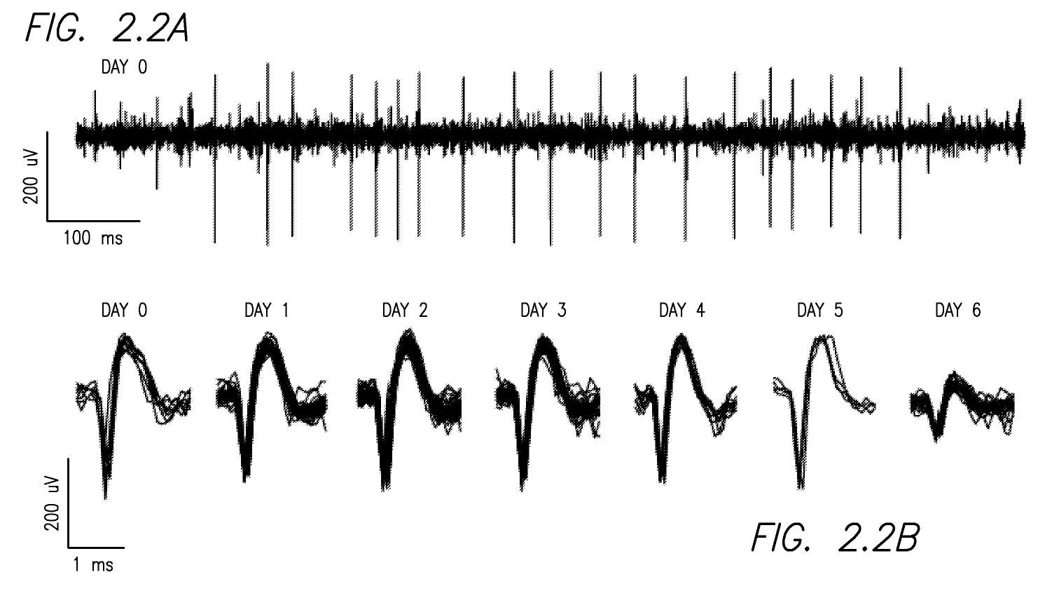

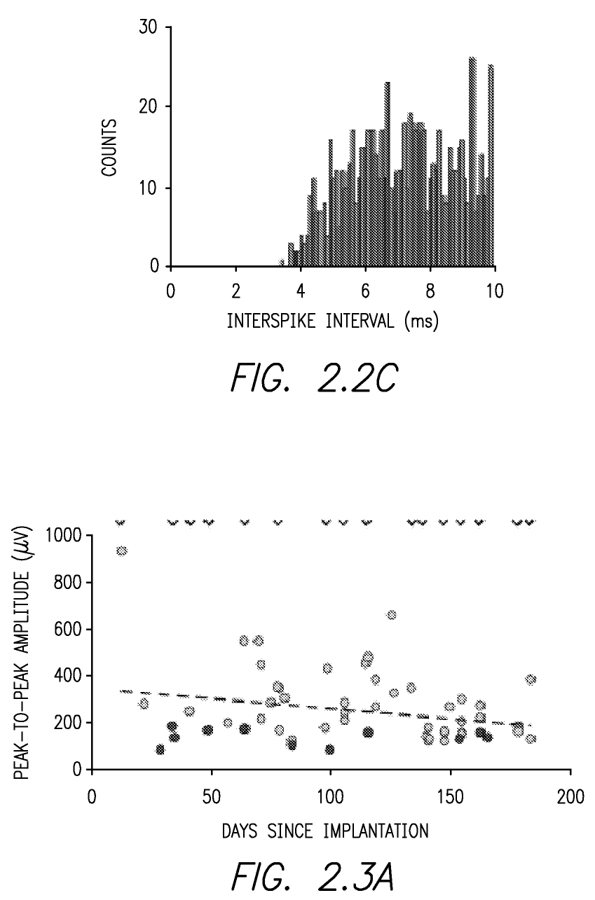

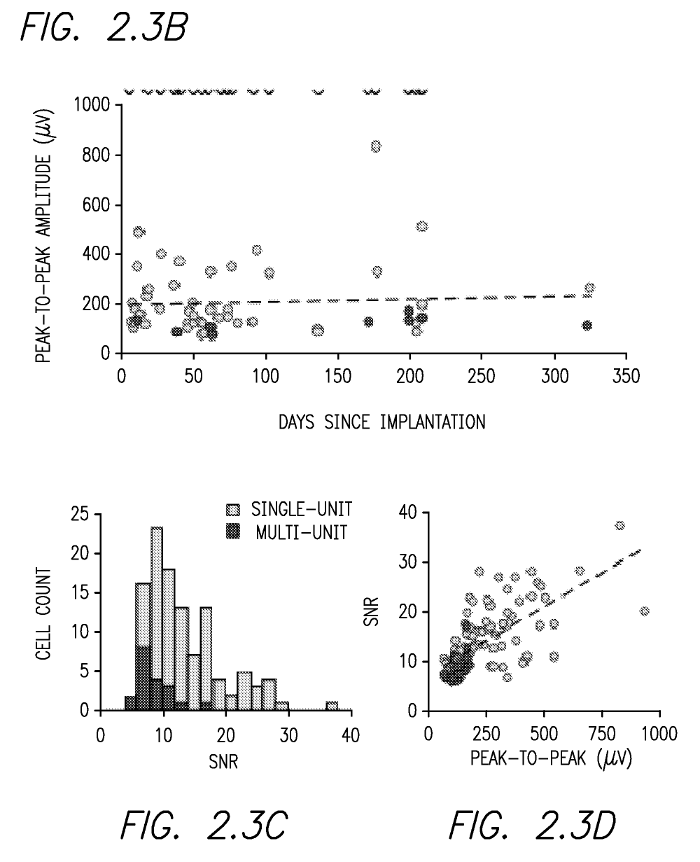

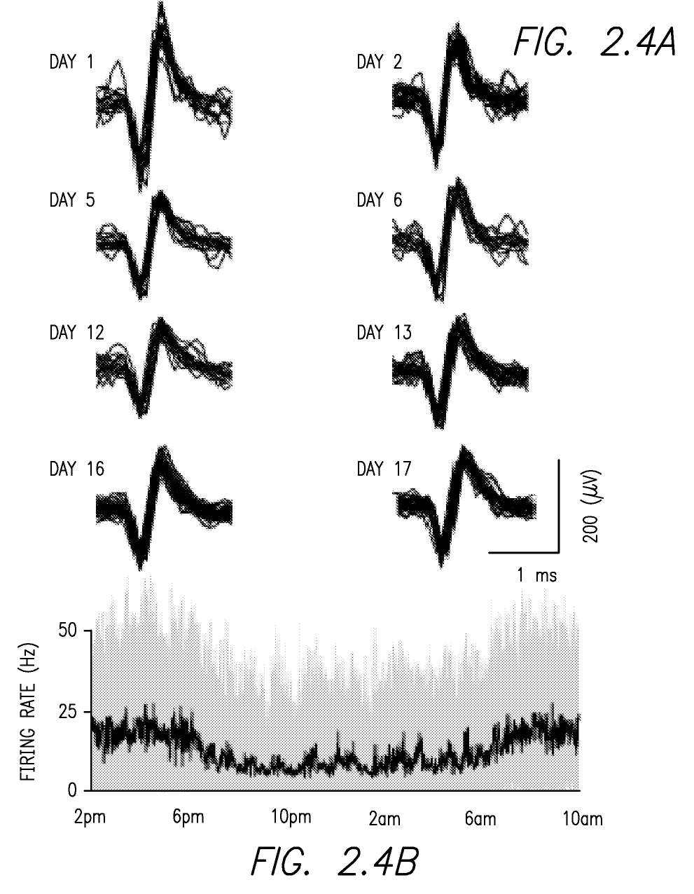

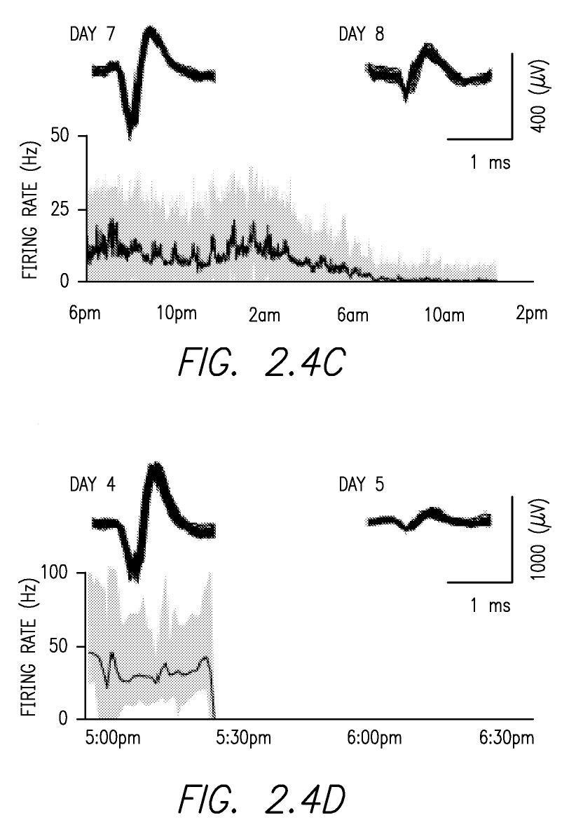

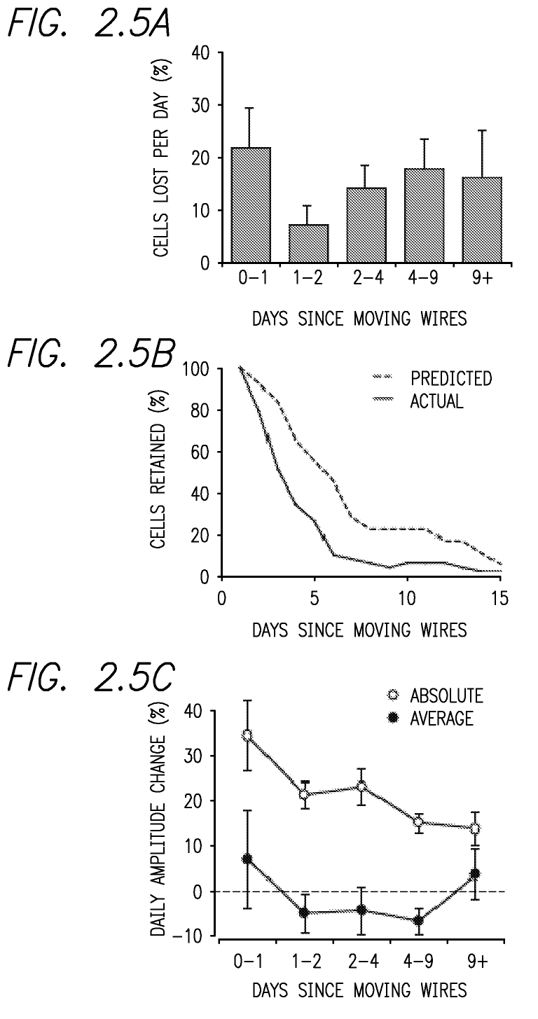

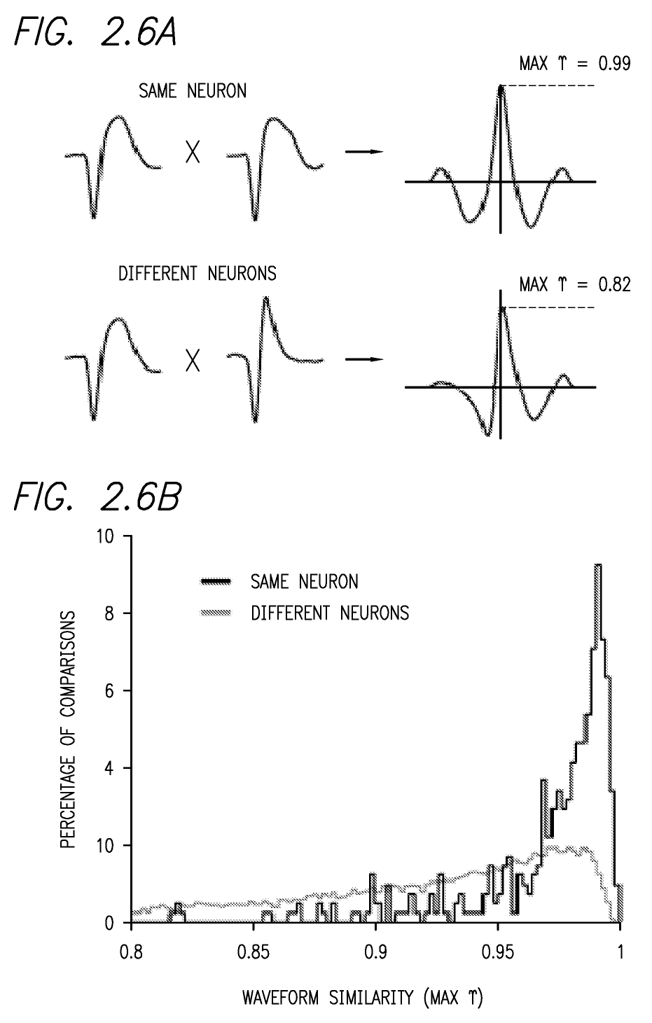

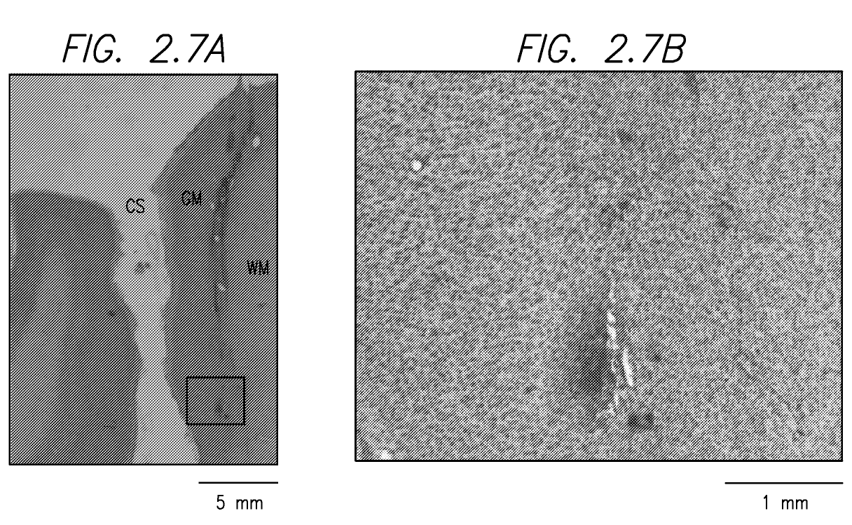

Implant construction. The microwire implant consisted of two components constructed separately: an array of guide-tubes to align the microwires and a connector block for making electrical contacts. FIG. 2.1 shows the assembly procedure. The guide tubes were made from 40-mm lengths of polyamide tubes with 225-μm internal diameter (part #822200, A-M Systems Inc., Carlsborg, Wash.) threaded onto tungsten rods and aligned in the desired spatial arrangement. This was performed by taping the ends of the rods to a piece of card with a cut-out window (FIG. 2.1A). Two rows of six tubes were taped to each side of the card to produce a 6×2 array. Once all the tubes lay parallel they were fixed at one end with a small amount of dental cement. After this had hardened, the rods at one end were splayed and more dental cement was applied. The result was a fan-shaped arrangement of tubes with a spacing of approximately 2 mm along one side and 300 μm along the other (FIG. 2.1B).

Centiloc-series pin connectors (part #031-9540-000, ITT Cannon, Santa Ana, Calif.) were crimped to 15-cm lengths of tungsten microwire (part #795500, A-M Systems Inc., Carlsborg, Wash.) and slotted through holes in a 4×3-way plastic connector block (part #CTA4-IP-60, ITT Cannon). The connector was held upside-down with the wires aligned in parallel down one side by threading them through holes in a spacer and weighting each with an alligator clip (FIG. 2.1C). Slow-setting epoxy was applied to the base and side of the connector to insulate the contacts and fix the wires. The connector was then rotated by 90°) so the bent wires projected from the side of the connector, where Silastic was applied to add strain relief (FIG. 2.1D,E).

At this stage, both components were sterilized by overnight (minimum 10 hours) immersion in freshly-activated glutaraldehyde sterilization fluid (Cidex Plus, Johnson and Johnson Medical Products); the tungsten rods were removed while the array was submerged to draw fluid into the interiors of the guide-tubes. The final assembly process was completed under aseptic conditions with sterile tools. After flushing with sterile water, each guide-tube was filled with antibiotic cream (Gentak, Akorn Inc., Buffalo Grove, Ill.) by injection through a 26 gauge needle placed around it. The tubes were then trimmed to a final length of around 20 mm and attached to the connector block with dental cement. The wires were threaded into the guide-tubes with the aid of magnifying loupes, and quick-setting two-part Silastic (Kwik-Sil, WPI Inc., Sarasota, Fla.) was applied to seal each end. Finally the microwires were cut to the appropriate length with sharp scissors perpendicular to the long axis, yielding a tip impedance of around 0.5 MΩ at 1 kHz, and retracted so that they protruded only slightly from the Silastic. The assembled implant is shown in FIG. 2.1E.

Implant surgeries. All procedures were approved by the University of Washington Institutional Animal Care and Use Committee (IACUC). Arrays were implanted in two male macacca nemestrina monkeys (monkey Y: 3 y/o, weight: 4.3 kg, and monkey K: 3 y/o, weight: 4.6 kg). Each animal received corticosteroids (dexamethasone 1 mg/kg PO) on the night before and at the beginning and end of surgery to reduce cerebral edema. Implantation was performed under aseptic conditions with inhalational anesthetic (isoflurane 2-2.5% in 50:50 O2:N2O). Heart rate, blood pressure, temperature, end-tidal CO2 and blood oxygen saturation were continuously monitored to ensure stable anesthesia, and fluids were administered via an intravenous catheter. With the animal in a stereotaxic frame, the skin and periosteum over the skull were resected and a craniotomy approximately 10 mm wide was drilled with a dental burr at co-ordinates A: 13 mm, L: 18 mm. A skull screw was placed close to the craniotomy to anchor the microwire connector block. Prior to opening the dura mater, the monkey was hyperventilated slightly to reduce the intracranial pressure. The dura mater was then resected to the edge of the craniotomy, permitting visualization of the central sulcus. Stimuli were delivered through a silver ball electrode placed anterior to the central sulcus to locate the area from which movements of the hand could be elicited with the lowest threshold. To reduce the relative movement of the brain and skull and stabilize recordings, the pia mater was bonded to the edge of the craniotomy with cyano-acrylate glue following the method described by Kralik et al. (2001). The microwire array was then lowered into position on a stereotaxic carrier such that the ends of the guide-tubes rested just above the pial surface over the hand representation, and oriented such that extruded wires would travel along the bank of the precentral cortex. The connector block was securely anchored to the skull screw with dental cement (FIG. 2.1F), and each wire was individually inserted into the cortex by grasping the exposed loop with fine, angled forceps and advancing slowly at a speed of approximately 1 mm/s. Where possible, penetration of the pia mater was verified through a microscope. We did not record from the wires at the time of surgery, preferring to position them slightly below the surface and advance them further at a later stage under ketamine anesthesia when M1 firing rates are robust. With the dura removed and the guide-tubes positioned just above the pial surface, the individually-inserted wires easily penetrated the brain without the problems of electrode buckling or tissue dimpling that have been reported with other methods (Swadlow et al. 2005; Kralik et al. 2001). However it is important to ensure that each wire has penetrated the pia at the time of surgery, since the formation of scar tissue may prevent insertion at a later stage. Once all microwires had been inserted, the craniotomy was tightly filled with gelfoam and sealed using dental cement. Care was taken to apply the cement initially in small quantities to avoid heating the tissue. A cross-section of the implanted microwire array is shown in FIG. 2.1G.

The titanium casing was attached with further skull screws and cement. To seal the inside of the casing, exposed skull was covered first with a layer of varnish (Copaliner, Bosworth, East Providence, R.I.) and then with a thin layer of dental cement. The layer of varnish proved useful in preventing fluid leaking from the skull into the casing, which could occur when only dental cement was used. Finally, the skin was drawn around the implant with several sutures. The entire procedure took approximately five hours and was followed by a full program of analgesics (buprenorphine 0.15 mg/kg IM and ketoprofen 5 mg/kg PO) and antibiotics (cephalexin 25 mg/kg PO).

Implant maintenance. Every 1-2 weeks the monkeys were lightly sedated with ketamine (10 mg/kg IM) in order to sterilize the inside of the head casing using warmed, dilute chlorohexadine solution followed by isopropol alcohol. If recording quality had deteriorated, or the current sample of cells had been sufficiently characterized for experimental purposes, we took this opportunity to move some or all of the microwires to find new cells. We typically moved between 4-8 wires during one of these sessions, aiming to achieve a sample of 2-5 cells. Our experiments required monitoring individual cells for several days each, so this sample was sufficient for a week or more of data collection. Once an appropriate sample of cells was obtained, we avoided moving additional wires to minimize the disturbance to the tissue.

While listening to an audio monitor of the recorded signal, the wires were nudged up or down with fine, angled forceps. Care was taken to avoid bending the wires while pushing them down, as kinks could prevent free movement through the guide-tubes. By grasping the wires only a short distance (no more than a few millimeters) above the top of the guide-tube, buckling or kinking of the wires and large, uncontrolled movements could be avoided. Although there is potential for the forceps to damage the insulation around the wires, in our design any breaks at the level that the wires are grasped will remain above or within the sealed guide-tubes, preventing any shunting of the recorded signal through the tissue.

Often the first movement of a wire after it had remained in place for days or weeks produced large changes in the audio quality, possibly as the tips broke through glial encapsulation. Subsequent movements did not produce such abrupt changes, although an increase in background noise often preceded the appearance of clean neurons. Unlike isoflurane anesthesia, ketamine sedation is associated with considerable activity in M1. Many neurons fire bursts of action potentials up to 50 Hz followed by periods of quiescence with an overall rhythmicity of around 0.2-0.4 Hz (Steriade et al. 1993), making it possible to position the wires at suitable depths to record units.

Recording. The recordings documented here were obtained with our Neurochip technology which has been described in detail previously (Mavoori et al. 2005; Jackson et al. 2007). Briefly, this battery-powered electronic implant amplifies, filters and samples the signal from a single microwire. For these recordings, the gain was set in the range ×1500-×12000, the filter pass band was 500 Hz-5 kHz and the sampling frequency was 11.7 ksp/s. The Neurochip recorded the raw signal, the rate of spike events accepted by its on-line dual time-amplitude window discriminator algorithm or some combination of both. Most of the analysis reported here is derived from sections of raw data recorded during the day-time while the monkeys performed a trained wrist movement task. Overnight records of firing rate were compiled with 100 ms bins and interspersed with 22 ms sections of raw signal every 3 minutes to assess cell stability. Changes in spike size during overnight recordings were verified during the following day-time session using a fresh battery (and often a different Neurochip circuit) to ensure these did not arise from non-stationarities in the electronics. During periods of trained behavior, the precise times of discriminated spike events were transmitted in real-time via infrared to a remote PC. Inter-spike interval (ISI) histograms compiled from these records were used to distinguish single-unit and multi-unit activity. Because transmission of data in this way requires an unbroken line of sight to the implant, we were unable to compile ISI histograms for overnight recordings during unrestrained behavior.

Post-mortem histology. At the end of the recording period in monkey Y, a surgical level of anesthesia was induced with sodium pentobarbitone (25 mg/kg IV) prior to perfusion through the heart with neutral-buffered formalin. 50 μm coronal sections of cortex were stained with Cresyl violet. Monkey K is still alive at the time of writing.

Analysis. Off-line, spike events were extracted from the daily raw recordings using the same discrimination algorithm as was implemented in real-time by the Neurochip. This algorithm comprised an adjustable threshold and two time-amplitude windows (Mavoori et al., 2005). Appropriate discriminator parameters were chosen for each day's record to compensate for some changes in spike waveform. In general we have found that the recording quality using our Neurochip system is excellent; the short leads to the Neurochip circuit do not pick up interference or generate movement artifacts, and the battery power supply and titanium shielding effectively isolate the recording from other sources of electrical noise and artifacts. Therefore waveforms with peak-to-peak amplitudes greater than around 100 μV could usually be isolated on the basis of threshold crossing alone, although the subsequent time-amplitude windows were useful for rejecting the occasional erroneous trigger or, in rare cases, separating multiple cells present on the same electrode. We separated multiple cells only when the waveforms were clearly distinct (i.e. the respective time-amplitude windows did not overlap) to avoid artificially inflating signal-to-noise measures with overly restrictive discrimination criteria.

Cell identities were verified across multiple days of recording using several factors. We looked for stability of cell waveforms and behavioral tuning assessed both during a trained wrist-movement task and unrestrained behavior (Jackson et al. 2007). Continued identity of those cells for which overnight Neurochip recordings revealed gradual changes in waveforms was accepted so long as ISI histograms indicated that there was only one cell in the recording. If spike waveforms changed abruptly, or when a large change in waveform and/or behavioral tuning occurred on channels that were not tracked using the Neurochip the cell was considered to have been lost.

Accepted waveforms consisted of sections from 5 sampling points before to 15 sampling points after the threshold crossing (1.7 ms total). Cell size was quantified by averaging peak-to-peak amplitudes across accepted waveforms. In addition, for comparison with a previous report we calculated signal-to-noise ratio (SNR) using the technique described by Suner et al. (2005). In this method, accepted spike waveforms are aligned at the threshold crossing and the peak-to-peak amplitude, A, of the average waveform is calculated. Noise, ε, is quantified by the standard deviation of the residuals remaining after this average is subtracted from each individual waveform, and SNR is calculated as A/ε. Because this calculation relies on precise alignment of the individual waveforms to the threshold crossing, we performed the analysis on data that had been up-sampled by a factor of 4 using low-pass interpolation (MatLab interp function) to give an effective sampling resolution of 47 ksp/s. Other interpolation methods yielded similar results.

Finally, to evaluate the similarity in spike shapes we calculated linear correlation (r) values between time-shifted average waveforms (i.e. the cross-correlation function calculated between mean-subtracted, variance-normalized spike shapes). The maximum r value across time-shifts was used to quantify similarity, with a value of 1 indicating identical spike shapes irrespective of absolute amplitude differences. Similarity scores were calculated between all waveforms for the same cell recorded on different days, and between all pairs of different cells recorded during the experiment.

### Results

Dataset. This analysis is based on 113 cells (monkey Y: 58 cells, monkey K: 55 cells), of which 88% were verified as clean single-units from refractory periods in ISI histograms. This probably represents an underestimate of the maximum cell count that could have been obtained, since the single Neurochip recording channel and other experimental considerations meant we could not record from every microwire during every session. However, on the first day after the wires were moved we sampled all channels showing good activity; subsequently we typically followed each cell overnight with the Neurochip for several days to characterize its properties. Therefore these totals are a realistic estimate of the yield of usable neurons that can be expected under similar experimental conditions.

Spike amplitudes and signal-to-noise ratio (SNR). FIG. 2.2A shows a 1-second section of signal recorded from a microwire that had just been repositioned in M1 (subsequently referred to as day 0). This recording from monkey K was made 77 days after the initial microwire implant surgery. The burst of action potentials was typical of the robust activity seen under ketamine anesthesia, and the peak-to-peak amplitude (300 μV) is representative of well-isolated neurons obtained with this implant. Recordings on subsequent days with the monkey awake (FIG. 2.2B, days 1-5) show that the spike shape remained generally stable. On day 6 this large waveform was absent, and although smaller spikes were present in the recording, it is not possible to determine whether these arose from the same or a different neuron. The large waveform disappeared abruptly (towards the end of day 5), so it seems likely that movement of the electrode caused the cell to be lost or injured. The ISI histogram in FIG. 2.2C was compiled from data recorded while the monkey made repeated trained wrist movements on day 5. The absence of short intervals confirms that these waveforms were produced by a single cell (similar results were also obtained for the earlier days).

FIGS. 2.3A, 2.3B plot the peak-to-peak amplitude of all cells recorded in the two animals. Tick-marks above each graph indicate occasions when some of the microwires were repositioned under sedation. Combining data across both animals, wires were moved on 39 occasions to yield 113 units at an average of 2.9 cells on each occasion (range 1-9). In monkey Y the amplitude of recorded waveforms tended to decrease over time following array implantation, although this trend did not reach significance (Pearson's r=−0.25, two-tail P=0.06) and is partly the result of a single large-amplitude cell present in the first recording sessions with this array. During the final weeks of the 184-day experimental period, we were still able to record clean units with amplitudes up to 380 μV. In monkey K the amplitudes showed no significant change over a 326-day period (r=0.06, P=0.6). As expected, the amplitudes for waveforms that were dassed as multi-unit (dark grey, mean 125 μV, SD 32 μV) tended to be smaller than for single-units (light grey, mean 248 μV, SD 158 μV), although a few recordings with spike amplitudes up to 180 μV failed to yield cleanly discriminated single units. FIG. 2.3C shows a histogram of SNR values for all cells classified as single- or multi-unit. Most multi-unit recordings had low SNR values, although one ISI histogram indicated multiple cells despite discriminated waveforms with a SNR of almost 17. In this case, the recording probably included two cells with large but similar spike waveforms. Such occurrences underline the importance of compiling ISI histograms to verify clean discrimination rather than relying on SNR alone. SNR values for clean units were generally high (mean 14.5, SD 6.2) and across the population were positively correlated with peak-to-peak amplitudes (FIG. 2.3D, r=0.68, P<1e-6).

Stability of recordings. Clean single-units could often be followed up to several weeks after positioning the microwires. FIG. 2.4A shows sample waveforms from one of the most stable cells, which was followed for a period of 17 days. Using the on-board Neurochip electronics, we were able to monitor the firing rate of this cell continuously as the monkey moved freely around the home cage. Interspersed sections of raw recording were used to assess the stability of spike waveforms. FIG. 2.4B shows a 20-hour record of firing rate covering a period from 16 to 17 days after this microwire had been moved. Mean firing rate decreased during the night, but this drop was not associated with a change in action potential and rates returned to original levels the next morning. ISI histograms compiled from spike events transmitted in real-time during trained behavior, combined with the stability of directional tuning (Jackson et al., 2007) strongly suggest that we were recording the same single-unit throughout this period. The size and shape of the action potential waveforms remained consistent despite the energetic behavior of the monkeys in the home cage, which included frequent jumping, somersaulting, swinging upside down and sudden movements of the head.

Although the majority of datasets tracked stable activity, in some cases the cell was lost during recording with the Neurochip. FIG. 2.4C shows firing rate data collected for one cell between 7 and 8 days after moving this microwire. The recorded rate of spike events classified by the Neurochip declined steadily from 2 am onwards. At this point, the monkey was probably asleep, given the periodic fluctuations in firing rate characteristic of sleep cycles (Jackson et al., 2007). Interspersed raw recordings revealed that the decline in detected spike events was caused by a steady reduction in waveform amplitude, such that by 6 am very few spikes satisfied the criteria for discrimination. The amplitude of this cell continued to decline and by day 9 could not be distinguished above the background noise. This suggests that a slow drift or change at the electrode-tissue interface caused steady signal degradation over several days. A different example of an unstable recording is shown in FIG. 2.4D. At around 5:30 pm on day 4 the rate of discriminated events on this channel fell abruptly to zero. Although small waveforms could subsequently be seen in the recording, it is not possible to know whether any arose from the same cell. Of 26 neurons lost while the Neurochip was recording, the majority (20) declined steadily in amplitude. The remaining 6 disappeared abruptly and all these cases of sudden loss occurred during the day-time. This suggests that abrupt disappearances may be related to movements of the animal, consistent with a previous report (Santhanam et al. 2007). On one occasion the abrupt loss of a cell coincided with observation of the monkey shaking his head vigorously.

For 69 cells that were followed over consecutive recording sessions we calculated the percentage lost per day (FIG. 2.5A). The highest rate of loss (23% or 7/31 cells) occurred immediately after the wires were moved (i.e. between day 0 and day 1). This rate fell to 7% (4/53 cells) between days 1-2, before rising to between 15-20% per day over the next few weeks. By combining cumulatively the loss rates from day 1 onwards, we predicted the proportion of day 1 cells that would be retained on each subsequent day. This prediction, shown as a dashed line in FIG. 2.5B, suggests that approximately half of the original population of day 1 neurons would be retained after one week and only one tenth after two weeks. FIG. 2.5B also shows the actual proportion of cells that were recorded on each subsequent day (solid line), which falls slightly below the predicted retention rate because we did not always follow every cell until it was lost. Microwires were sometimes moved to sample new cells even if previously documented neurons were still present. Those cells which were not followed over consecutive recording sessions were not included in the calculation of loss rates.

The average percent change in peak-to-peak amplitudes of retained cells over successive recordings is shown as solid circles in FIG. 2.5C (these values exclude cells that were lost between sessions). On average this was not significantly different from zero over any period tested, suggesting that declining spike amplitudes for some cells were balanced by increases in the amplitude of other cells, particularly between days 0-1. The open circles in FIG. 2.5C plot the average absolute change (i.e. disregarding sign), which decreased steadily over each day after repositioning of the microwires (r=−0.67, P=0.002). This may be in part because some unstable cells were lost progressively from the sample, although even those cells that were retained tended to show the largest amplitude changes during the first few days after the wires were moved.

To characterize the similarity between spike shapes recorded from the same cell on different days we calculated the maximum linear correlation coefficient between time-shifted average waveforms (FIG. 2.6A). This similarity score is independent of the absolute amplitudes of waveforms, and a value of 1 indicates identically-shaped spikes. Waveforms for the same neuron on different days tended to be very similar with 81% of r values greater than 0.95, compared with only 37% of similarity scores calculated between waveforms of different cells (FIG. 2.6B). Although this suggests that in general spike shapes remain consistent, the overlap between the distributions shown in FIG. 2.6B indicates that spikes from different cells can often exhibit similar waveforms. Hence a consistent spike shape alone does not provide conclusive evidence that the same individual neurons are present in recordings on subsequent days.

Post-mortem histology. Cresyl-stained slices from monkey Y revealed clear electrode tracks running down the anterior bank of the central sulcus along the edge of the grey/white matter border (FIG. 2.7A). Dense glial scarring surrounded these tracks and electrode tips (FIG. 2.7B). The track orientation revealed by this histology helps explain the successful yield and large waveform amplitudes obtained during this experiment, as many tracks ran close to layer V where large pyramidal cell bodies are located. For this reason moveable microwire arrays may be particularly applicable to recording down the banks of sulci where cells can be found at different depths.

### Discussion

Suitability of moveable microwire arrays for long-term recording. Moveable microwire arrays provide a favorable combination of high signal-to-noise, excellent neuronal stability during free behavior and long-term performance. Large-amplitude waveforms can be acquired consistently upon moving the microwires into fresh tissue (at the time of writing, the implant in monkey K is still recording clean neurons after 18 months) and the moveable wires allow recording from multiple sites down the banks of sulci. Although the yield of simultaneously recorded neurons is relatively low, individual cells remain well-isolated and stable for many days at a time (one cell remained consistent for 3 weeks before the microwire was moved). This technique is therefore well-suited to studying the neural correlates of natural, unrestrained behaviors and processes that extend over periods of several days, for example learning. The implant is simple to construct with available components and sufficiently small to be used in conjunction with implanted electronic circuitry for wireless recording. For our experiments, the array was enclosed by a large (6 cm-diameter) chamber which also housed our electronics and battery. In other situations a smaller implant about the size of a conventional recording chamber would be feasible, or multiple arrays could be implanted to record from different cortical areas. These microwires can also be used to deliver intracortical microstimulation.

Moveable microwires may be especially useful for experiments that require repeated monitoring of small population of cells over periods of several days, for example to study neural plasticity during learning. In such cases, reliability in identifying specific cells may be critical to unambiguous interpretation of the data. In our experiments, a range of corroborating evidence supported the general consistency of cell identities through the recordings, including clean ISI histograms, stability of waveforms and consistent behavioral correlations over several days. However, no single test can provide a conclusive assurance that individual cells on any given day are the same as were recorded previously. Spike amplitudes could vary considerably over a 24 hour period and in many learning experiments a change in behavioral tuning may be the observation of interest. Different cells can often have similar waveforms (FIG. 2.6B) such that a consistent spike shape from day to day is no guarantee of identity. In some cases (e.g. pyramidal tract neurons), antidromic identification by stimulating output pathways may be useful in this regard (Lemon 1984) although the stability of antidromic latencies and thresholds over many days remains to be demonstrated. In light of these issues, continuous recording with implanted microprocessors or telemetry systems may prove valuable for future experiments revealing as it does both gradual and abrupt changes in spike shape.

Comparison with alternative techniques. The range of commercially available chronic electrode and microdrive designs has expanded considerably in recent years. Performance of the moveable microwire arrays can be compared meaningfully with fixed electrode arrays (Nicolelis et al. 2003; Suner et al. 2005; Santhanam et al. 2007) and miniature microdrives carrying conventional sharp electrodes (Wilson et al. 2003; Cham et al. 2005; Gray et al. 2006). The number of electrodes in high-density implants such as fixed microwires (Nicolelis et al. 2003) or the Utah array (Suner et al. 2005) can yield large cell counts, and these devices may be sufficiently small and safe for clinical use as BCIs. Long-term performance continues to improve, with spike recordings reported several years after implantation (Sandler et al. 2005; Suner et al. 2005). Stability of individual cells recorded on the Utah array over periods of several days has recently been demonstrated using an implanted recording system (Santhanam et al. 2007). Amplitude variations of a similar magnitude to the present study were reported. However, comparison with published data for both fixed microwires and Utah arrays suggests that the moveable microwires yields recordings with substantially higher SNR. Suner et al. (2005) reported a mean SNR value of 4.8 for signals ranked as ‘high quality’ on a Utah array, while Nicolelis et al. (2003) reported a mean SNR of 5.5 for fixed microwire recordings. By contrast, the mean SNR value for our moveable microwire recordings was 14.5. This improvement is also evident in the mean peak-to-peak amplitude (248 μV) as compared with fixed microwires (115 μV; Nicolelis et al. 2003). Furthermore, a single well-isolated unit was most often obtained with our method, as compared with the multi-unit recordings often obtained with fixed arrays. Although it remains to be seen whether single- or multi-unit data will be most appropriate for BCI applications (Carmena et al. 2003), isolating individual cells has clear benefits for scientific studies. Even if waveforms are sufficiently distinct to be separated, the presence of multiple cells on a channel may compromise the reliability of identifying neurons over several days, particularly given the changes of amplitude that can occur.

One explanation for the high SNR and prevalence of single-units on these electrodes may be the ability to adjust accurately their depth to position the tips within layer V, close to the large pyramidal cells. The ability to repeatedly advance individual electrodes into fresh tissue is also likely to be an important factor. A number of groups are working to reduce the tissue response to chronically implanted electrodes (Retterer et al. 2004; He et al. 2006) and the data reported here may offer incentive by demonstrating that significant future improvements in signal-to-noise performance should be possible.

In many ways the converse of these considerations applies to miniature microdrives fixed to the skull (Wilson et al. 2003; Cham et al., 2005; Swadlow et al., 2005; Gray et al. 2006). Such systems yield well-isolated units with good signal-to-noise upon initially penetrating the cortex, and can reach deeper structures, including the banks of sulci. However, reports on the long-term stability of individual neurons in primates are limited to several days at most (Wilson et al. 2003; Gray et al. 2006). In an attempt to address this issue, Cham et al. (2005) have proposed an automated tracking algorithm for continually adjusting electrode depth to maintain cell isolation, although the practicality of this remains to be proven. Stability could possibly be improved using flexible microwires as in our moveable array instead of rigid, sharp electrodes, but this would likely require resection of the dura mater in primates. Commercially available screw-drives such as the Neuralynx microdrive (Neuralynx, Bozeman, Mont.) may prove suitable for positioning microwires in primate cortex if the risk of infection can be managed, perhaps using the combination of antibiotic cream and Silastic described in this report. The use of such screw-drives would be advantageous in situations requiring calibrated depth measurement. However, in practice the precise depth control of a microdrive may not confer sufficient long-term advantage to justify the increased implant size. In our experience, many cells changed size or were lost in the first 24 hours after moving the microwires, so efforts to optimize specific waveforms on day 0 were often unrewarded, while channels with small waveforms subsequently yielded stable, well-isolated cells a day later.

### REFERENCES CITED IN EXAMPLE 2

1. Baker S N, et al. J Neurosci Methods 94: 5-17, 1999.
2. Biran R, et al. Exp Neurol 195: 115-126, 2005.
3. Carmena J M, et al. PLoS Biol 1: E42, 2003.
4. Cham J G, et al. J Neurophysiol 93: 570-579, 2005.
5. Eckhorn R. and Thomas U. J Neurosci Methods 49: 175-179, 1993.
6. Gray C M, et al. Soc Neurosci Abstract 148.16, 2006.
7. Griffith R W, and Humphrey D R. Neurosci Left 406: 81-86, 2006.
8. He W, et al. J Neural Eng 3: 316-326, 2006.
9. Jackson A, et al. Nature 444: 56-60, 2006a.
10. Jackson A, et al. IEEE Trans Neural Sys Rehab Eng 14: 187-190, 2006b.
11. Jackson A, et al. J Neurophysiol 97: 360-374, 2007.
12. Johnson J L, and Welsh J P. Methods 30: 64-78, 2003.
13. Kralik J D, et al. Methods 25: 121-150, 2001.
14. Lemon R N. Methods for neuronal recording in conscious animals. In: IBRO Handbook Series: Methods in Neurosciences 4, edited by A D Smith. London: Wiley, 1984, p. 1-162.
15. Mavoori J, et al. J Neurosci Meth 148: 71-77, 2005.
16. Nicolelis M A L. Nature Reviews Neuroscience 4: 417-422, 2003.
17. Nicolelis M A L, et al. PNAS 100: 11041-11046, 2003.
18. Nordhausen C T, et al. Brain Res 726: 129-140, 1996.
19. O'Keefe J, and Recce M L. Hippocampus 3: 317-330, 1993.
20. Retterer S T, et al. IEEE Trans Biomed Eng 51:2063-2073, 2004.
21. Sandier A J, et al. Soc Neurosci Abstract 402.8, 2005.
22. Santhanam G, et al. IEEE Trans Biomed Eng in press.
23. Schwartz A B, et al. Neuron 52: 205-220, 2006.
24. Steriade M, et al. J Neurosci 13: 3252-3265, 1993.
25. Suner S, et al. IEEE Trans Neural Syst Rehabil Eng 13: 524-541, 2005.
26. Swadlow H A, et al. J Neurophysiol 93: 2959-2965, 2005.
27. Szarowski D H, et al. Brain Res 983: 23-35, 2003.
28. Wilson F A, et al. J Neurosci Methods 127: 49-61, 2003.
29. Wilson M A, and McNaughton. Science 261:1055-1058, 1993.

### Example 3

### Epidural Conditioning Paradigm

The demonstration of plasticity in Example 1 involved invasive recording of single motor cortex cells through wires implanted in the cerebral cortex, which is technically challenging and clinically problematic for implementation in human subjects. This example provides a strategy to avoid limitations of long-term recording stability from single cells and the risks of infection or damage due to invasive recording procedures. By using epidural conditioning, the dura mater covering the brain remains intact. The surface brain potentials are recorded non-invasively through the dura, and likewise the cortex is stimulated through the dura. Recent findings indicate that the electrocorticogram [ECoG], recorded from the dural surface, has high-frequency components that are essentially equivalent to recording the activity of multiple underlying neurons. The cortex can also be readily stimulated by electrodes on the surface of the dura. This suggests that the plasticity phenomenon can be replicated with epidural recording and stimulation, an approach that will be significantly more practical for human application.

Epidural procedures involve implanting a grid of ECoG electrodes over the motor cortex of subjects. The electrode grid typically consists of up to 64 contacts [Hollenberg et al, J. Neurosci Meth., 153: 147-153, 2006]. First, testing of the paradigm can be performed with primate subjects as follows. The output effects evoked from stimulating all sites of the grid are documented with the monkey seated in a primate chair, as described in Example 1 above. Evoked output is measured in the form of electromyographic recordings from forearm muscles, and by isometric forces generated about the wrist. Two of the grid electrodes that evoke different outputs are selected for recording [R] and stimulating [S], the rest serving as controls for non-contingent changes. The recordings from the R electrode provides the high-frequency ECoG activity that is processed by the Neurochip and converted to stimuli delivered through electrode S. This activity-dependent stimulation is continued for 24-48 hours, and the outputs evoked from the sites are tested for changes analogous to those observed with intracortical recording and stimulation. This testing protocol can be used to optimize efficacy of epidural conditioning procedures to strengthen connections between R and S.

### Example 4

### Electromyography (EMG) Derived Signals

A second alternative to invasive single unit recording in the conditioning paradigm is recording the electromyographic [EMG] signal from associated muscles. The neural activity of motor cortex sites controlling a muscle is tightly correlated with the EMG of specific associated muscle. The strength of these correlations makes it possible to use the EMG signal as a surrogate of the activity in the associated cortical sites for the conditioning procedure.

The use of EMG signals can be optimized by replicating the study described in Example 1 with pulses triggered from the EMG rather than from cortical units. An array of wire electrodes can be tested for outputs evoked by cortical stimulation as described above. A muscle with particularly strong effects evoked from one of the wires [called R] can be chosen as the surrogate for R. EMG electrodes are chronically implanted in this “R muscle” and led subcutaneously to the cortical brain computer interface (BCI) for triggering stimuli at another site [S], chosen for having a different evoked response. Again, the conditioning paradigm is implemented for 24-48 hours and the sites tested for changes in output. Successful conditioning paradigms obtained with the implanted wire electrodes can be tested with epidural stimulation with a less invasive grid of ECoG electrodes.

In addition to creating long-term plastic changes, the EMG-triggered stimulation of the cortical site of muscle representation will also boost the efficacy of the cortical signal in generating movement. This positive feedback loop augments contraction of weak muscles and optimizes the parameters for this mode of therapy.

### Example 5

### Hebbian Strengthening of Corticospinal Pathways

This example describes a chronically implanted artificial connection from brain to spinal cord that can be incorporated into normal motor function. The example also describes Hebbian strengthening of corticospinal pathways that can be produced by spinal stimuli delivered synchronously with cortical activity. This provides a novel method of restoring compromised circuitry in the injured spinal cord.

Volitionally controllable motor cortex neurons are used to generate functional electrical stimulation of spinal cord. The action potentials of motor cortical cells trigger the stimulus pulses directly or through a simple transform. The recurrent BCI continuously delivers neurally controlled stimulus pulses under free behavioral conditions to test the monkeys' ability to incorporate this novel “connection”. A second consequence of stimulating spinal cord synchronously with cortical cell spikes is the enhancement of efficacy of corticospinal connections.

Cortical control of functional electric stimulation of spinal cord. Paradigm: Cortical units are recorded from an implanted wire array in arm area of motor cortex and the activity of a well-isolated unit is detected by the BCI. A set of 8-12 wires is implanted in cervical spinal cord, using methods described by Mushahwar et al (2000, Exp Neurol 163: 422-9), and the output effects evoked from the spinal sites are documented. The R-BCI is then used to convert action potentials of a motor cortical cell to trigger stimuli at a spinal site that produces a well-defined response on wrist movement. Initially, a cell is chosen that is activated synergistically with the evoked movement, but cells with different response properties can also be used. The stimulus strength is initially below threshold for evoking movements at rest, but supra-threshold for output effects from cell bursts, during volitional movement. First, the effect of closing the R-BCI loop while the monkey performs the 2-D wrist task [flexion-extension and radial-ulnar deviations] is documented. Then the wrist torques, and activities of forearm muscles and the cortical cell, are recorded, and the tuning of the cell and muscles during isometric torques about the wrist in the 2-D plane are documented. Several sessions of performance on the 2-D task both with and without connecting the R-BCI are performed to document changes produced by this connection. The R-BCI remains disconnected between these sessions.

Next, continuous operation of the R-BCI loop from cortical cell to spinal site is performed. The appropriate stimulus intensity is derived in the above phase. The activity of the cell and relevant muscles throughout unrestrained movements are stored on the BCI memory chip and downloaded daily for off-line analysis of the monkey's motor behavior and changes in the mode of activating the cell. This provides the first method by which the biological connections between motor cortex cells and spinal cord can be compensated with artificial connections via a continuously operating implanted recurrent BCI. The method also provides for a prosthetic application of the R-BCI to assist in movements in partial spinal cord injury. Patients can learn to compensate for impaired corticospinal connections or spinal cord injury through such an implanted R-BCI allowing cortical cell activity to directly evoke or facilitate movement.

Long-term potentiation of corticospinal pathways. Paradigm: To demonstrate potentiation of corticospinal pathways by spike-triggered stimulation of spinal sites, the muscle responses and isometric wrist torques evoked by repetitive stimulating through the cortical and spinal electrodes are measured with the monkey seated at rest. Responses are documented for several [4-8] days before and after chronic operation of the corticospinal R-BCI for 1-3 days. For each site the amplitudes of the stimulus trains [100 ms at 300/sec] are fixed at the levels just suprathreshold for evoking movements prior to conditioning. Increased responses reveal long-term potentiation of corticospinal pathways analogous to those seen in cortex. In this case the Hebbian mechanism could involve potentiation of terminals of pyramidal tract neurons synchronized with the triggering cell and carrying action potentials arriving on spinal INs and motoneurons activated by the stimulation. Several additional relay sites in cortex or brainstem could also mediate changes. As controls, the outputs evoked from the other cortical and spinal electrodes are documented, to confirm that the changes in output are specific to the two interconnected sites.

Recording the intraspinal field potential responses evoked from single cortical stimuli delivered at each of the spinal sites and from all cortical wires can be used to further document the extent and specificity of changes in corticospinal connections produced by the conditioning paradigm. Once it is established that spike-triggered spinal stimulation produces changes in output effects and/or spinal fields, one can repeat the conditioning procedure with longer delays between action potentials and stimuli to test the time-dependency of the mechanism. When this was done with the cortico-cortical R-BCI, the conditioning effect was not obtained with delays greater than 50 ms, indicating a time-dependent Hebbian reinforcement mechanism. To implement the same control for the corticospinal connections, the PSoC software is used to deliver the same number of stimuli as spikes, but temporally delayed.

We expect that continuous operation of the spike-triggered stimulation via the corticospinal R-BCI will lead to changes in the movements and the spinal field potentials evoked from the cortical recording site due to potentiation of pyramidal tract terminals and/or other possible relays. No changes in evoked responses are expected from control sites in cortex and spinal cord that are not included in the R-BCI conditioning. Similar to previous observations in cortex, no changes are expected in the output effects evoked from the spinal sites.

This work establishes that the plasticity seen in the cortex with the R-BCI can also be demonstrated for pairs of cortical and spinal sites, with greater spatial separation. Potentiation of the corticospinal pathway produced by continuous operation of the R-BCI indicates a significant new therapeutic modality for strengthening surviving connections in spinal cord injury.

### Example 6

### Electromyography (EMG)-Contingent Conditioning

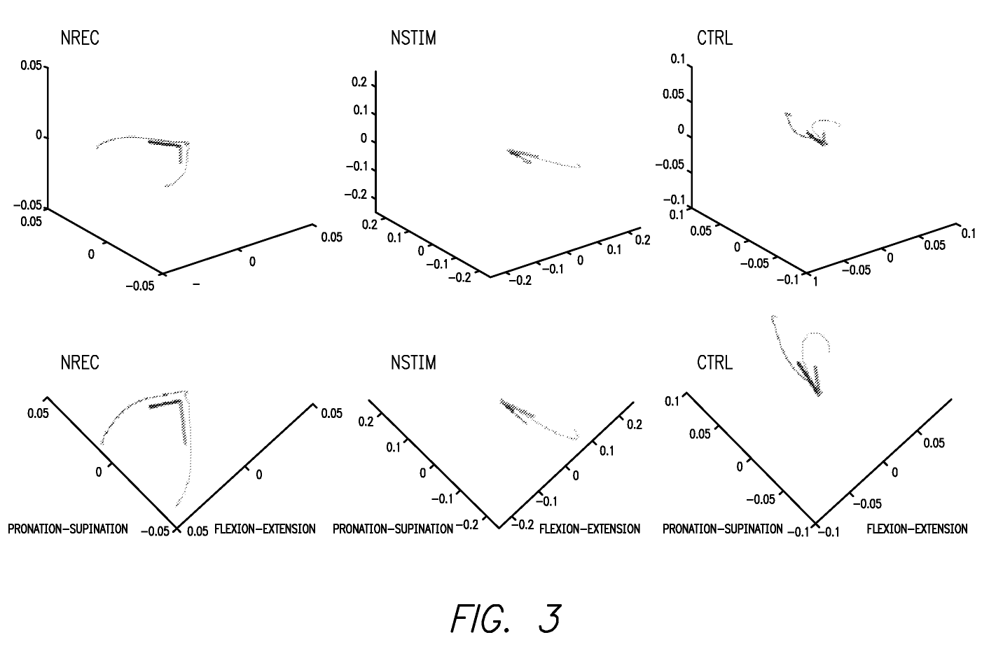

This example demonstrates the efficacy of conditioning contingent on EMG activity and the feasibility of indirectly detecting cortical spike activity via muscle activity recorded in an electromyogram. FIG. 3 illustrates the cortically evoked responses as measured by isometric torque vectors about the wrist. [Upper set shows torques in 3 directions: radial-ulnar (z axis), flexion-extension (x-axis) and pronation-supination (y axis); lower set shows same data plotted for last two dimensions.] Responses were evoked by trains of microstimuli at three cortical sites: Nrec, corresponding to the recorded muscle that generated stimulus triggers during conditioning; Nstim, corresponding to the site that was stimulated during conditioning; and Ctrl, an unrelated control site. The blue lines represent baseline torque vectors prior to conditioning. The green lines represent torque vectors after 8 hours of conditioning. The stimulation protocol during these 8 hours was 1:1 for discriminated emg ‘spikes’ to stimulus pulses, with 0 delay. Stimulation intensity was 38 microamps, with a 4 ms post-spike refractory period and stimulus rates of 72.761 +/−75.330 Hz, with peak rates around 250 Hz during active forelimb movement. The salient point is the post-conditioning shift in the vector for the Nrec site toward the vector at the Nstim site, and the absence of change from the other two sites. This replicates the conditioning effects documented in Example 1 above for cortical recording and stimulation.

### Example 7

### Direct Control of Paralyzed Muscles by Cortical Neurons

A potential treatment for paralysis resulting from spinal cord injury is to route control signals from the brain around the injury via artificial connections. Such signals could then control electrical stimulation of muscles, thereby restoring volitional movement to paralyzed limbs. In previously separate experiments, activity of motor cortex neurons related to actual or imagined movements has been used to control computer cursors and robotic arms, and paralyzed muscles have been activated by functional electrical stimulation (FES). This example shows that monkeys can directly control stimulation of muscles using the activity of neurons in motor cortex, thereby restoring goal-directed movements to a transiently paralyzed arm. Moreover, neurons could control functional stimulation equally well regardless of any prior association to movement, a finding that significantly expands the source of control signals for brain-machine interfaces. Monkeys learned to utilize these artificial connections from cortical cells to muscles to generate bidirectional wrist torques, and controlled multiple neuron-muscle pairs simultaneously. Such direct transforms from cortical activity to muscle stimulation could be implemented by autonomous electronic circuitry, creating a relatively natural neuroprosthesis. These results are the first demonstration that direct artificial connections between cortical cells and muscles can compensate for interrupted physiological pathways and restore volitional control of movement to paralyzed limbs.

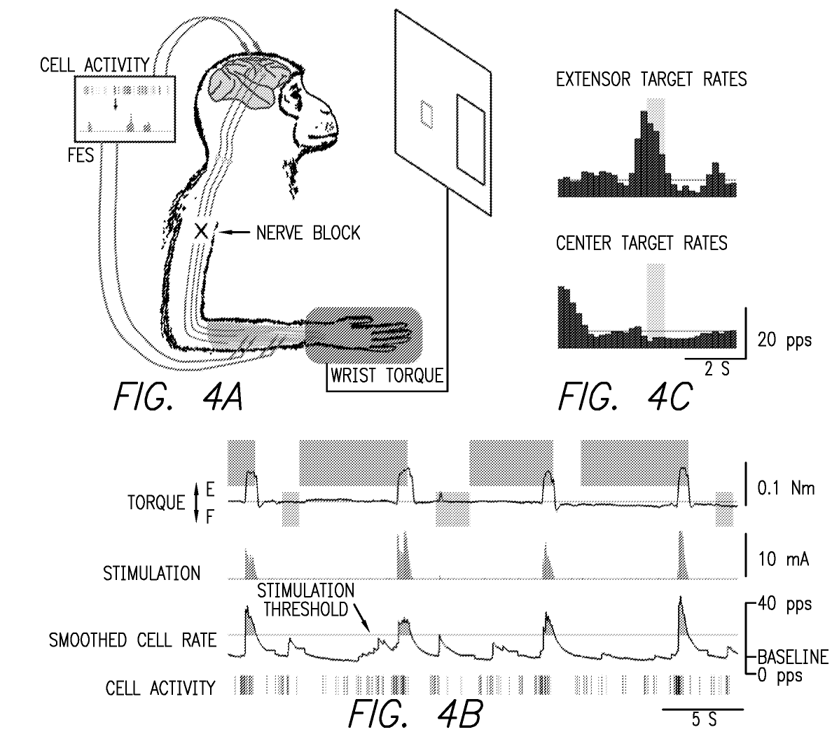

Spinal cord injury impairs neural pathways between the brain and limbs, but spares both the motor cortex and muscles. Recent studies have shown that quadriplegic patients, could volitionally modulate activity of neurons in hand area of motor cortex, even several years after paralysis, and that monkeys could use cortical activity to control a robotic arm to acquire targets and feed themselves. These and other brain-machine interface studies used sophisticated algorithms to decode task-related activity of neural populations and calculate requisite control parameters for external devices. An alternate strategy to restore limb function is to directly connect cortical cell activity to control stimulation of a patient's paralyzed muscles (FIG. 4A). Here we show that monkeys can learn to use direct artificial connections from arbitrary motor cortex cells to grade stimulation delivered to multiple muscles and restore goal-directed movement to a paralyzed arm.

In previous biofeedback studies monkeys rapidly learned to control the discharge rates of newly isolated neurons in motor cortex to obtain rewards. We used a similar operant conditioning paradigm for single neurons in hand and wrist area of motor cortex of two monkeys. We tested volitional control of cell activity by displaying smoothed discharge rate as cursor position on a monitor and rewarding the monkeys for maintaining activity within randomly presented high- or low-rate targets. The directional tuning of most cells was also characterized in an isometric 2-dimensional wrist tracking task. However, our experiment employed all sufficiently well-isolated cells encountered, with no selection bias for possible association to movement or directional tuning.

Monkeys demonstrated volitional control of the discharge rates of nearly all cells tested within the first 10-minute practice session. Although cell activity controlled the cursor directly, monkeys often continued to produce wrist torques during these initial sessions (FIG. 8). We then blocked peripheral nerves innervating the wrist muscles with a local anesthetic. Despite loss of motor function and sensory feedback from the innervated forearm, monkeys continued to control the cursor with cell activity for 45 of 46 cells after the nerve block. FIG. 8 shows the loss of flexor and extensor torques following injections of local anesthetic, while the monkey continued to volitionally control the cell activity. The nerve block was confirmed by the monkey's inability to perform the 2-dimensional torque tracking task.

We then converted cell activity into proportional stimuli delivered to paralyzed muscles. The cursor was now controlled by wrist torque, and the monkey was rewarded for maintaining FES-evoked torque within peripheral and center (i.e., zero-torque) targets for 0.5-1.0 s. To allow the monkey to grade contraction force, stimulation current was made linearly proportional to cell rate when the cell discharged above a threshold.

The example in FIG. 4B shows a monkey modulating cell activity to control FES and generate appropriate torques via paralyzed wrist extensor muscles. The monkey learned to increase cell activity to activate the stimulator and acquire the extensor targets, and to maintain activity below the stimulation threshold to relax the muscle and acquire the center targets. Both monkeys were able to control muscle FES during nerve block and acquire torque targets with 44 of the 45 cells tested (5 cells from monkey 1 and 39 from monkey L).

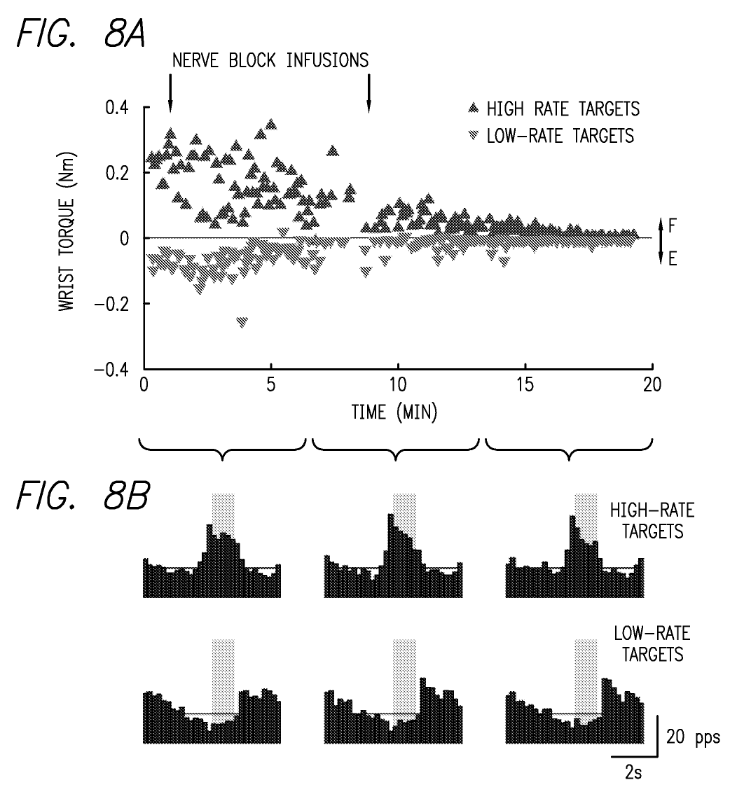

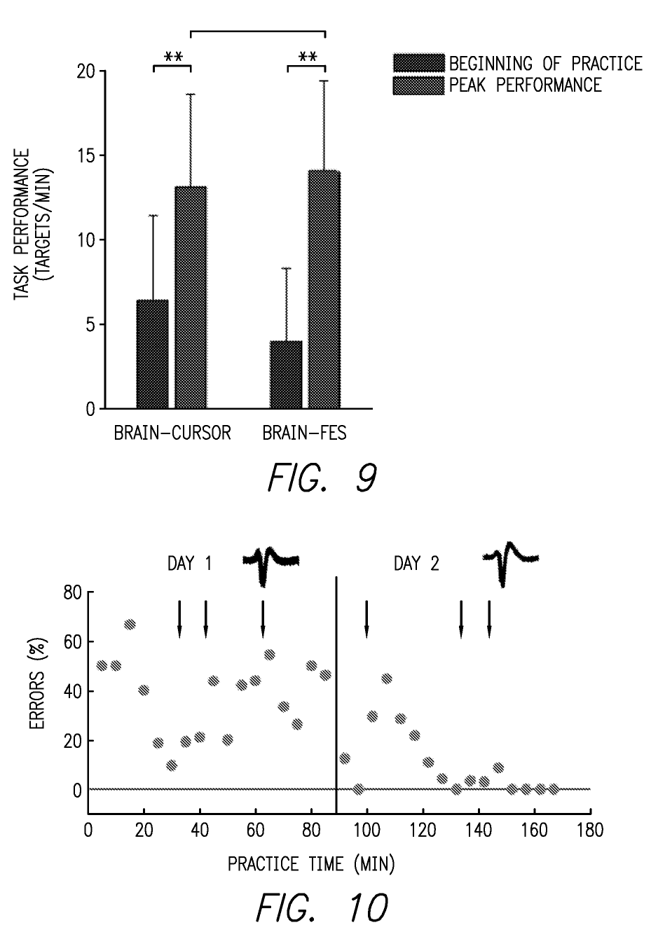

For each cell the monkeys' control improved with practice, as evidenced by more rapid acquisition of targets and fewer errors. Monkeys began using cell activity to control the stimulator almost immediately, and improved substantially during the relatively brief practice sessions with each cell (mean duration 66 min). To quantify improvement we compared performance during the initial two minutes of practice and during the two-minute period with the highest performance, typically just before task difficulty was increased to probe the limits of FES control. The rate of target acquisition with FES control was over three times greater during peak performance (14.1±5.3 torque targets acquired/minute; mean±SD) compared to the beginning of practice (4.0±4.3 targets/min; p<0.001; FIG. 9). Peak target acquisition rates during brain-controlled FES were similar to those seen when cell activity controlled the cursor directly before nerve block (13.2±5.5 targets/min; p=0.66).

With continued practice monkeys also learned to control the torque more precisely with cell activity, making fewer target acquisition errors and more often acquiring targets on the first attempt. A target acquisition error was defined as triggering the stimulator to acquire the peripheral target when the center target was displayed. Monkeys made target errors on only 0.8±5.1% of targets during peak performance for each cell compared to 20.7±28.9% of targets at the beginning of practice (p<0.001; FIG. 10). They also made 81% fewer failed attempts to acquire the target during peak performance (0.10±0.31 failed attempts per target) compared to the beginning of practice (0.52±0.93; p<0.001).

To test whether FES could also be controlled by decreases in cell activity, we set stimulation current to be inversely proportional to cell rate below a threshold for 11 cells. Monkey L learned to control stimulation with this inverse relation just as well as with a positive relation between cell rate and stimulus current (38 cells, some tested in both groups; p>0.46), acquiring 13.4±3.9 targets per minute and making no errors during peak performance.

The activity of a single cell could also be used to control stimulation of antagonist muscle groups and restore bidirectional movements. FIG. 2 shows an example of one cell that controlled stimulation of flexor muscles with high discharge rates and extensor muscles with low rates. The monkey learned to control cell activity and grade contraction force to rapidly satisfy targets at five different torque levels. The nerve blocks remained very effective, as evidenced by negligible torques produced in either direction when the stimulators were turned off during target presentation (FIG. 9). Seven cells tested with such bidirectional control performed similarly to cells that controlled only one muscle group, although target acquisition rates were marginally slower (9.8±3.7 targets/min; p=0.06).

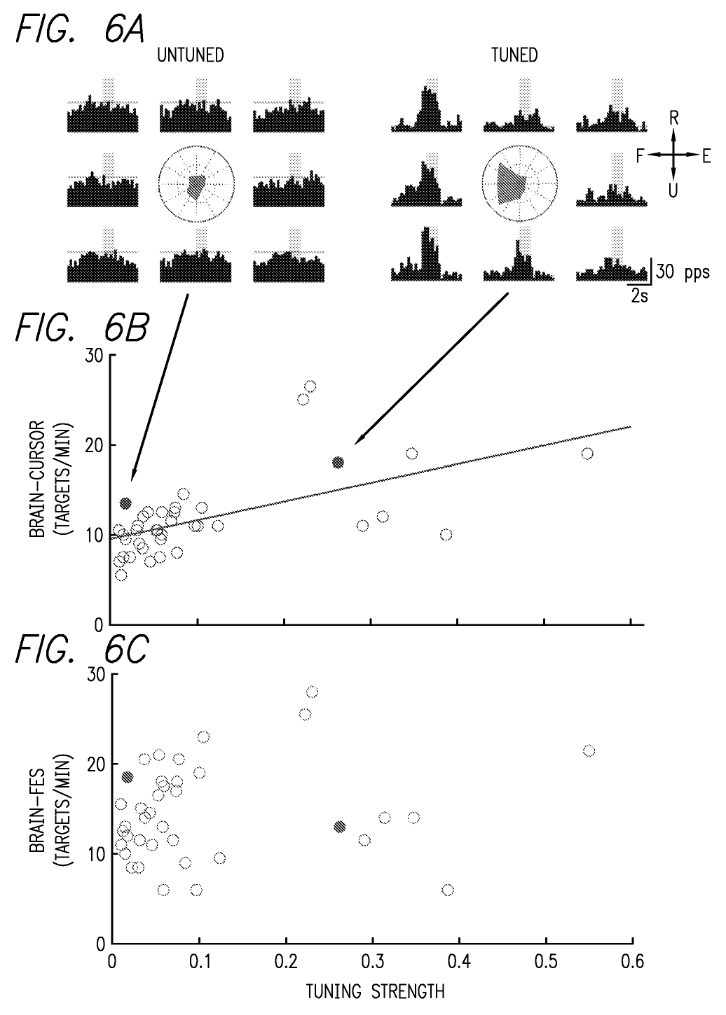

The assumptions underlying common neural decoding schemes would predict that monkeys should be able to control FES torque better with cells that are strongly related to wrist movements than with unrelated cells. To investigate this, we documented cell activity during a 2-dimensional wrist tracking task before the nerve block, and calculated the directional tuning for each cell (FIG. 6A). The magnitude of directional tuning did correlate significantly with the monkeys' ability to bring the cursor into the optimally placed targets with cell activity during the initial 10-minute practice period (r2=0.33, p<0.001; FIG. 6B). However, cell tuning was not a good predictor of the peak target acquisition rates during subsequent brain-controlled FES (r2=0.03, p=0.33; FIG. 6C). For example, with the untuned cell on the left in FIG. 6A the monkey acquired 18.5 targets per minute. The tuned (n=9) and untuned (n=29) cells showed no difference in any measure of FES control (target acquisition rates, errors, or failed attempts; p>0.51).

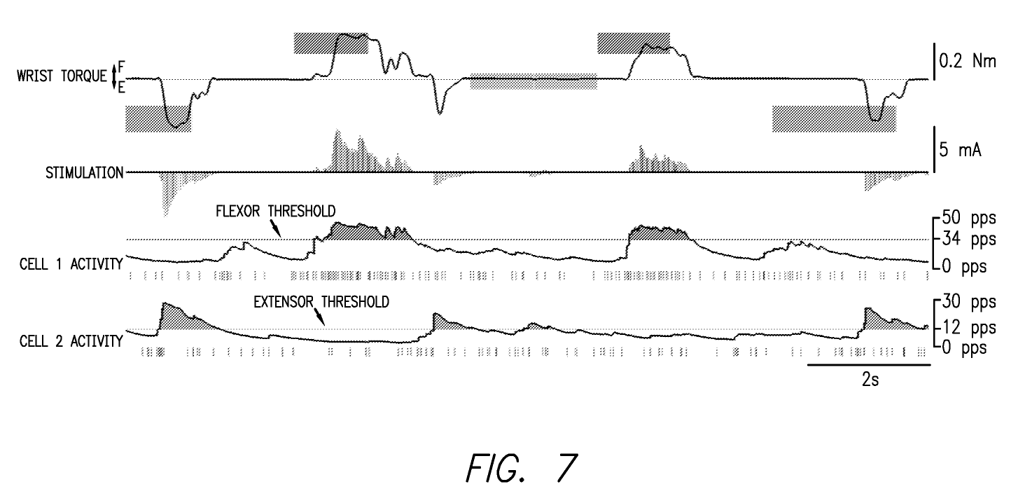

Extending the strategy of direct neural control to more complex movements will require additional control signals. As a first step toward this goal, we tested a monkey's ability to simultaneously control two cell-muscle pairs. FIG. 7 shows monkey L using high discharge rates of one cell to control FES of flexor muscles and high rates of a second cell to control extensor muscles. The monkey learned to independently modulate the activity of five cell pairs in order to control antagonist muscles and rapidly acquire bi-directional torque targets at rates similar to single cells (11.6±3.8 targets/min, p=0.32).

These findings have several implications for future approaches to neuroprosthetic control. In contrast to the conventional strategy of deriving control signals from the combined activity of a neural population, it may prove efficacious to maintain separate signal pathways from cells to muscles. Using direct channels from single cells to specific muscles may provide the brain with more distinguishable outcomes of the cell activity and allow innate motor learning mechanisms to help optimize control of the new connections. The brain's ability to adapt to novel but consistent sensorimotor contingencies has been amply documented, and motor cortex can adapt rapidly to learn new motor skills. Motor circuitry can compensate for drastic changes in connectivity, such as surgically cross-connected nerves controlling wrist flexor and extensor muscles, or targeted reinnervation for control of prosthetic limbs.

Our finding that monkeys could learn to use virtually any motor cortex cell to control muscle stimulation, regardless of the cell's original relation to wrist movement (FIG. 6C), suggests another advantage of directly tapping single cell activity. Strategies based on decoding the activity of neural ensembles to obtain movement parameters or muscle activity depend on finding cells that modulate sufficiently with the output variables during actual or imagined movements. Instead, arbitrary cells available on recording arrays could be brought under volitional control using biofeedback, substantially expanding the source of control signals for brain-machine interfaces. This and previous biofeedback studies have shown that even cells with no discernable relation to muscles can be volitionally modulated after brief practice sessions. Issues concerning the use of individual cells and neural populations for prosthetic control are further discussed in the supplementary portions of this Example.

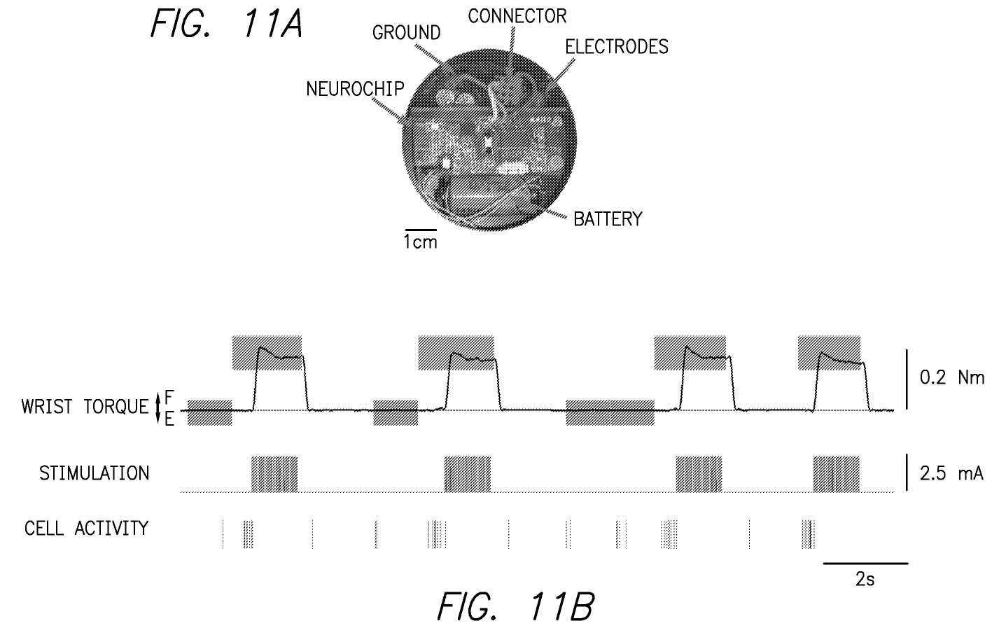

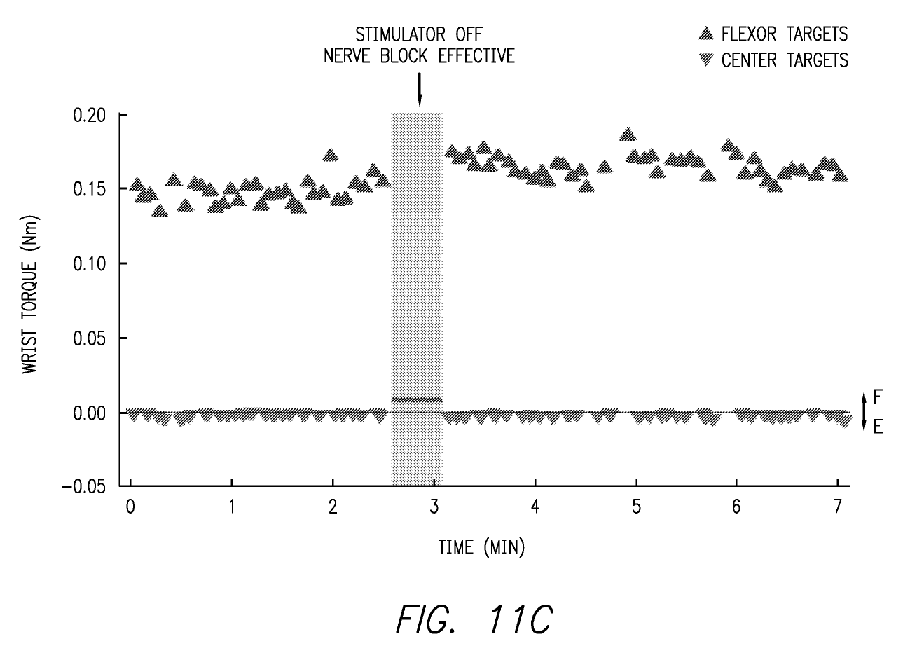

The degree of FES control demonstrated here was limited by the relatively brief training time provided by the transient nerve block. Implanted electronic circuitry will enable adaptive learning over much longer times and under more varied conditions. For example, the autonomous ‘Neurochip’ system can discriminate single cell activity and deliver stimulation through days of free behaviour. In several preliminary FES sessions, we confirmed that this system would allow a monkey to trigger stimulation of a paralyzed muscle with cell activity and acquire torque targets (FIG. 11). Such autonomous low-power circuits could permit subjects to practice continuously with an artificial connection from brain to muscles or the spinal cord, without requiring complex decoding algorithms or robotic arms. Further development of such direct-control strategies may lead to implantable devices that could help restore volitional movements to individuals living with paralysis.

### Methods

Subjects. Two male Macaca nemestrina monkeys participated in the experiments (4-5 years old, weight 4.5-6.5 kg). All procedures were approved by the University of Washington Institutional Animal Care and Use Committee.

Recording and paralysis. Activity of single motor cortex cells was recorded using either acute (Monkey I & L) or chronic (Monkey L) electrodes. Each session began by quantifying the cell's responses during an isometric, eight-target wrist torque tracking task. Volitional control of cell activity was confirmed by operantly rewarding acquisition of targets with a cursor controlled by cell rates. Wrist muscles were then paralyzed by injecting anesthetic (3% chloroprocaine or 2% lidocaine, each with 1:100,000 epinephrine) into catheters or cuffs surrounding the median, ulnar and/or radial nerves.

Brain-controlled FES. Cell activity controlled the intensity of stimuli delivered via bi-polar electrodes implanted in one or more paralyzed wrist muscles. When cell rate (smoothed over 0.5 s sliding window) crossed a threshold, biphasic constant-current stimuli (cathode-leading; 0.75-1.0 ms pulse width) were delivered at 50/s. For most cells, stimulus current was made proportional to cell rate above a threshold to allow the monkey to grade contraction force (e.g., stimulus current=0.1 mA×[cell rate−threshold]; to a maximum of 10 mA). Some cells controlled stimulation in inverse proportion to cell rate below a threshold.

Analysis. Strength of directional tuning was calculated for cells during the initial torque tracking task using the vector method (Batschelet, E. Circular Statistics in Biology, Academic Press, London, 1981). Peak performance was quantified by the maximum number of targets acquired during a two-minute period. Peak performance was compared among conditions and to performance during the initial two minutes of practice using the nonparametric ranksum test. Regression analysis determined correlations between directional tuning and peak performance during brain control of a cursor or FES.

Cortical recording. Sterile surgeries were performed with isoflurane anesthesia (1-1.5% in 50:50 02:N2O). All surgeries were followed by a program of analgesics (buprenorphine 0.15 mg/kg IM and ketoprofen 5 mg/kg PO) and antibiotics (cephalexin 25 mg/kg PO). Each animal was implanted with a cranial recording chamber over left hand and wrist area of motor cortex at stereotaxic coordinates A: 13 mm, L: 18 mm to permit cortical recordings (Evarts, E. V., 1968, J Neurophysiol 31, 14-27; Woolsey, C. N. et al., 1952, Res Publ Assoc Res Nerv Ment Dis 30, 238-64). To obtain longer duration cell recordings, monkey L was re-implanted with a chronic electrode array over left motor cortex. The array of 12 independently movable microwires is described in Example 2 above. Briefly, 50 μm tungsten wires were threaded through individual polyamide guide-tubes in a 2×6 array that was anchored to the skull. This array provided stable recordings from the same isolated cell for the duration of an experimental session, and across multiple days for ten cells (Jackson, A., et al., 2007, J Neurophysiol 97, 360-74).

Nerve block implant. Reversible paralysis of the right wrist was achieved with one of two nerve block methods. First, catheters were implanted in the brachial plexus, near cords giving rise to the radial, ulnar and median nerves. Epidural catheters (19 Ga., Arrow International) were inserted into the epineurium surrounding each nerve and anchored in place with cyanoacrylate. Second, nerve cuffs with catheters33 were implanted around the median and ulnar nerves in the upper arm. Catheters terminating in the lumen of each Silastic cuff (4 mm inner diameter, 30 mm long) permitted the nerves to be bathed in anesthetic. Nerves were identified by electrical stimulation, and catheters were tunneled subcutaneously to exit the skin on the upper back and sealed with an injection port. Thirty-one cells controlled FES during nerve blocks induced by the catheter method, and the remaining 13 cells during blocks induced by cuffs.

Experimental paradigm. The monkey sat with his right elbow and hand immobilized by padded splints while a transducer measured the flexion-extension (F-E) and radial-ulnar (R-U) torques produced about the wrist (see FIG. 4A). To receive an applesauce reward, the monkey maintained wrist torque within a center target (zero torque) followed by one of eight peripheral targets specifying different combinations of F-E and R-U torque (average magnitude 0.13±0.01 nM). Isolated cell activity was discriminated on-line using template-matching software (Alpha Omega MSD). Subsequently, cell activity controlled cursor movement in one dimension. Inter-spike intervals were averaged over a 0.5 s sliding window to create a continuous signal for cursor position (and later FES control). If the cell was directionally tuned, targets were aligned with its preferred direction. For untuned cells or cells without tuning information (i.e., cells isolated after nerve block began), either the left or right screen position was arbitrarily chosen to represent high discharge rates for visual feedback. Monkeys practiced cell control for 10 minutes, maintaining discharge rate within each target for 0.5-1.0 s to receive a reward.

Nerve block. We blocked nerves leading to wrist muscles with local anesthetic to create temporary motor paralysis. Block onset typically occurred after 5-60 minutes, depending on anesthetic and block method. During this time the monkeys continued to perform the cell-controlled target tracking task. Additional doses were given regularly to maintain paralysis during FES control.

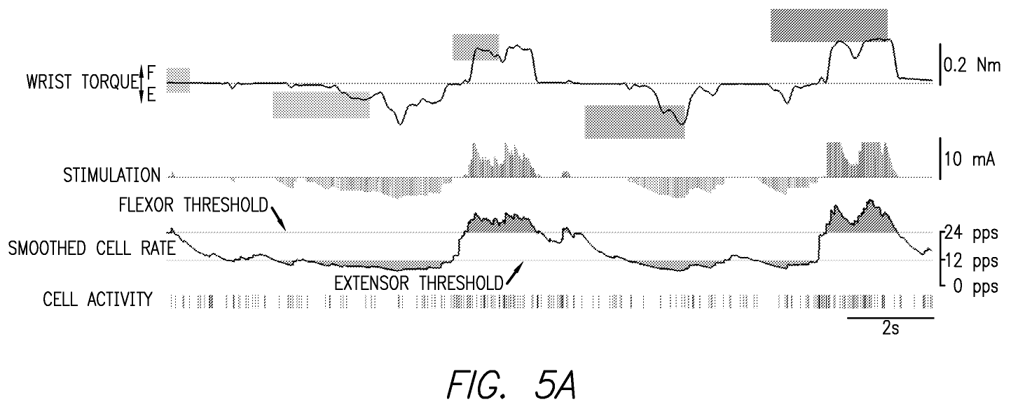

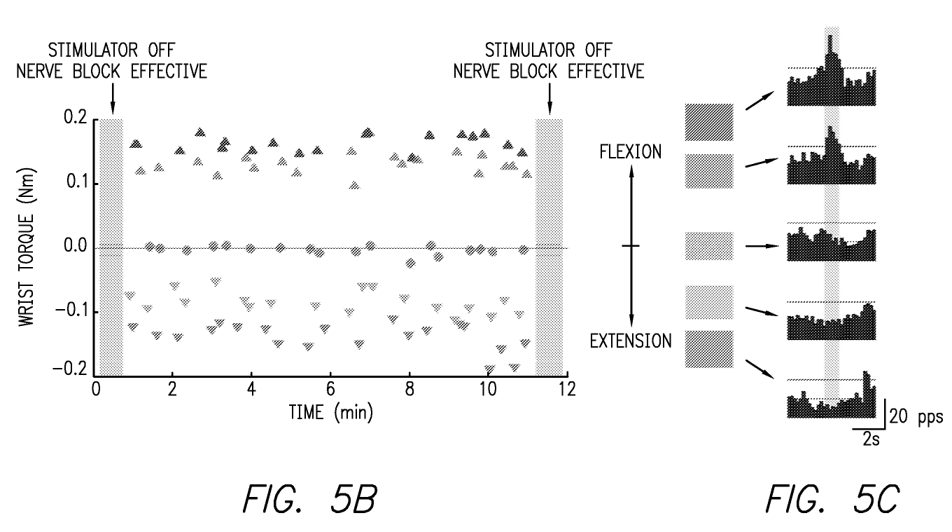

Brain-controlled FES. After onset of paralysis and an average of 36±22 minutes of cell-controlled target tracking, the cell activity was converted to stimuli delivered to one or more paralyzed muscles. Wrist torque controlled the position of the cursor, and targets were randomly displayed on the monitor in one dimension. Monkeys were required to maintain torque within each target for 0.5-1.0 s (mean 0.56 s) to receive a reward. Targets remained on the screen until satisfied, followed by presentation of the next target either immediately or after a 1.5 s reward period. Forty-two cells controlled stimulation current in proportion to cell rate, permitting the monkey to grade contraction force. Two of these cells also controlled stimulation via the autonomous ‘Neurochip’ to deliver a 1s train of stimuli (2.5 mA, 50/s) when smoothed cell rate exceeded a threshold (FIG. 11). Similarly, the first two cells in monkey I also triggered a 1s train of 50/s stimuli at 5 mA. To confirm continued nerve block during the practice session, the stimulator was turned off after every 10 minutes of FES to assure that the monkey could not acquire the peripheral target through volitional muscle contractions. FIGS. 5B & 11 illustrate the torques produced with the stimulator active compared to periods when the stimulator was turned off for 30 s. With the stimulator off, the monkey repeatedly attempted to satisfy the target but produced ≦10% of the torque used to acquire the active target. For all such test periods with each cell the monkeys produced an average maximum of only 18.0±21.3% of the torque used to satisfy the targets.

Data sampling. Signals were digitized and stored to disk for offline analysis. Raw recording from motor cortex was band-passed from 1-10 kHz and sampled at 25 kHz, along with spike times from the online discrimination. Wrist torques (flexion-extension and radial-ulnar) were sampled at 5 kHz, and smoothed and down-sampled to 500 Hz during offline analysis. We also recorded behavioural parameters (target on screen, etc.), and muscle stimulation amplitude and timing (5 kHz).

Data Analysis. Task difficulty was increased incrementally by raising levels of torque targets and increasing hold times. This complicated the quantification of skill learning. Improvements were evident as higher performance levels prior to increments in task requirements (e.g., FIG. 10), and these times were compared with performance at the beginning of a practice session. Specifically, the two-minute period with the peak performance was compared to the initial two minutes of practice (e.g., targets per minute). Control precision was measured by target errors and the number of failed attempts to reach a target. A target acquisition error was counted when the monkey activated the stimulator while the center target was on the screen, resulting in sufficient torque to satisfy the peripheral target had it been presented. Target errors are reported as the percentage of center targets presented. A failed attempt was counted whenever the monkey briefly acquired a peripheral torque target but did not satisfy the required hold time. A T-test was used to compare average torques during graded FES control. Otherwise, the non-parametric ranksum test was used for all comparisons as at least one data set for each remaining comparison failed Lilliefors test for normality. All reported values are means±standard deviation (SD).

### Supplementary Methods

Cortical recording. These studies employed all well-isolated active neurons encountered by the recording electrodes, without any selection bias for cells related to wrist movement. The consequent proportion of significantly tuned cells recorded (9/38; 24%) agrees approximately with Evart's original report that ≦31% of pyramidal tract neurons in motor cortex were clearly correlated with hand movement (Evarts, E. V., 1968, J Neurophysiol 31, 14-27). Location of sites in primary motor cortex was confirmed by intra-cortical microstimulation (ICMS) during preliminary mapping with acute electrodes to identify sites for low-threshold (10-50 μA) activation of forearm muscles. ICMS was not used during experimental sessions because stimulation prior to recording tended to suppress neural activity for long periods, and stimulus effects after the experiments were potentially compromised by residual nerve block. Postmortem examination of monkey I confirmed recording sites were in primary motor cortex; monkey L is ongoing.

Muscle stimulation. Bipolar stimulating wires were implanted surgically or transcutaneously in eight forearm muscles. Pairs of multi-stranded stainless steel wires (AS631 or AS637, Cooner Wire), bared of insulation for 0.2-0.5 cm, were inserted into the muscle belly ˜2 cm apart and parallel to muscles fibers. During surgical implantation (monkey I), wires were sutured to the epimysium. Wires inserted transcutaneously (monkey L) were secured to the skin with tissue glue (Vetbond, 3M) and porous tape (Transpore, 3M). Implantation in the following muscles was confirmed by responses to brief trains of stimulation: flexor muscles (digitorum superficialis, FDS; carpi radialis, FCR; carpi ulnaris, FCU; palmaris longus, PL); extensor muscles (digitorum communis, EDC; carpi radialis, ECR; carpi ulnaris, ECU; digitorum 4&5, ED4,5).

During experimental sessions, each cortical cell typically controlled simultaneous stimulation of two synergist muscles (e.g., wrist flexors or extensors). Four (of 44) cells controlled only a single muscle due to technical difficulties with the second stimulator. Seven cells also controlled stimulation of antagonist muscle groups, activating two synergist muscles (with high rates) and one (2 cells) or two (3 cells) antagonist muscles (with low rates) to produce bi-directional torques.

Recording sessions. Data are reported for 44 cells recorded during nerve blocks performed during 47 separate sessions (1 session/day). Nine cells were recorded during two consecutive sessions, and one cell was recorded during five sessions. In addition, during ten sessions, two cells were used to control FES either successively (2 cells) or simultaneously (5 cell pairs, with 3 pairs recorded over 2 sessions each).

Nerve block. During most sessions, both flexor and extensor wrist muscles were blocked. In some sessions, however, nerve block targeted flexor muscles (median and ulnar nerves) or extensor muscles (radial nerve), depending on the direction of cell control. For example, when a cell discharged high rates for targets in the extensor direction, the radial nerve was blocked. Focusing on flexors or extensors permitted the duration of nerve blocks to be extended with additional doses, while avoiding systemic reactions to the anesthetics (maximum total dose was 1 ml/kg). Comparing cells with complete nerve blocks of both flexors and extensors (n=25) to cells with only flexors (n=15) or extensors paralyzed (n=4) showed no difference in the monkeys' ability to use cell-controlled FES (p>0.30). During cell-controlled FES nerves were blocked by chloroprocaine for 33 cells and lidocaine for 11 cells.

Conversion of cell activity to FES. One of our goals was to test relatively direct transforms between cell activity and muscle stimulation. The most direct transformation is triggering a stimulus pulse from every cell discharge, but this proved problematic for several reasons. First, changing stimulus rate did not grade contraction force as effectively as changing pulse width or amplitude, which presumably changed the number of motor units recruited. Second, cell rates in excess of 40 pps are required to achieve fused tetanus when stimulating monkey forearm muscles directly, and few of the cells tested discharged above this rate for the duration of the target hold period. Finally, most of the cells had a non-zero baseline rate that would generate sustained low-frequency stimulation and produce muscle fatigue. These issues were resolved by imposing a discharge rate threshold to gate stimulation. We then delivered 50/s stimuli with intensities proportional to discharge rate exceeding (standard case), or falling below (inverse case), this threshold. For example, for the cell shown in FIG. 5, stimulation of the flexor muscle (FCU) was proportional to rate above a threshold of 24 pps (stimulus current=0.8 mA/pps×[cell rate−24 pps]; ≦10 mA) and stimulation of the extensor muscles (ECU & ED4,5) was inversely proportional to cell rate below a second threshold (stimulus current=0.6 mA/pps×[12 pps−cell rate]; ≦10 mA).

Unit discrimination amidst artifacts. The stimulation parameters permitted reliable discrimination of cortical cell activity despite stimulus artifacts present in the recordings. Stimulus artifacts were sufficiently brief to preserve ≧80% of simultaneous neural signals for spike discrimination, and their shapes were reliably separable from spike events. To minimize artifacts, stimuli delivered to two synergistic muscles were simultaneous.

Directional Tuning. To quantify directional tuning in the isometric flexor-extensor and radial-ulnar plane, vectors (Vi) were constructed in each of the eight peripheral target directions (θi) with magnitudes proportional to the average cell rate (ri) during the target hold period. These vectors were then summed and normalized to produce a resultant vector (V) with magnitude between 0 and 1 (equation 1).

$$V = \frac{\sum {V}_{i}}{\sum ‖ {V}_{i} ‖} \qquad ( 1 )$$

Larger resultant magnitudes indicate more sharply tuned cells. A bootstrap test was used to determine significant (p<0.05) directional tuning by assigning measured spike rates to random target positions 4,000 times. Based on this test, cells were considered tuned (n=9) or untuned (n=29). Regressions of tuning strength vs. performance were calculated for 38 of the 44 cells for which sufficient data were available.

Baseline activity. Baseline cell rates were obtained during one-minute periods in which the monkey viewed a blank screen immediately following the first 10-minute period of cell control of the cursor. Baseline rates were computed from average cell activity only over periods when the monkey produced no wrist torque.

Task performance. Quantification of performance during operant conditioning is complicated by the fact that task requirements were incrementally increased as skill improved over time for most cells. When the monkey learned to regularly acquire targets with a new cell, hold times were increased, target magnitudes were increased and/or target width reduced to make the task more challenging. Performance measures would not be expected to increase monotonically throughout the conditioning period (e.g., FIG. 10). There were periods of time, however, when performance reached a high level before the task was made more challenging. We used these times to quantify peak performance and improvement compared to the beginning of a practice session. In addition, stimulus current gain (current/spike rate) was sometimes adjusted to offset muscle fatigue and maintain a consistent relation between wrist torque and cell spike rate. Improvements in target error rates were not due to changing task difficulty or stimulus gain, as target parameters and gain were not significantly different between the initial and peak performance (p>0.25).

To probe the limits of FES control, the task was made more challenging for 27/44 cells in one or more of the following ways. Target hold times were gradually raised by an average of 0.34±0.31 s above a baseline value of 0.50 s (17 cells). Torque magnitude required to satisfy a target was increased by an average of 25% (0.03±0.01 nM; 17 cells), and target width was decreased by an average of 19% (0.07±0.04 nM; 17 cells). Task difficulty was not changed for cells recorded from monkey I (although target hold times of 1.0 s were used), or for the initial 12 cells recorded from monkey L (7 of which were recorded from acute electrodes permitting limited practice durations).

### Supplementary Results

Data from 44 cells (5 from monkey I and 39 from monkey L) were pooled across monkeys and recording techniques, since there were no differences in performance or directional tuning among groups. Specifically, there was no difference in FES control (i.e., target acquisition rates, failed attempts or target errors) achieved by the two monkeys (p>0.26). All cells in monkey I and 7/39 cells in Monkey L were recorded using acute electrodes. The remaining 32 cells in monkey L were recorded via a chronic electrode array. There was no difference in FES control via cells recorded on acute and chronic electrodes (p>0.43).

Practice times controlling the stimulator with each cell ranged from 2 to 317 minutes (mean 66 min; n=44) and continued until cell recordings were lost, the monkey became satiated, or the nerve block began to wear off. There was no difference in the strength of directional tuning among cells used to control stimulus currents proportionally or inversely to rate (p=0.29). In addition, the degree of modulation in cell activity to acquire the FES torque targets was similar for both types of stimulator control. The cells controlling inverse stimulation were modulated by 10.2±3.4 pps, while the cells controlling direct stimulation were modulated by 14.7±10.8 pps (p=0.14). Overall, cells modulated over a slightly greater range (14.3±10.3 pps) during FES control compared to activity during the unparalyzed tracking task (12.5±9.3; p=0.04). Cell modulation was greatest during brain control of a cursor before nerve block (19.2±11.4 pps; p<0.002).

Performance during the normal torque-tracking task without paralysis can be compared to brain-controlled FES performance. Before paralysis, monkeys acquired 30.6±1.9 targets/minute by producing 0.16±0.01 nM of flexion-extension torque. After paralysis, monkeys used single cells to control FES and acquired up to 14.1±5.3 targets/minute by producing 0.14±0.04 nM of flexion-extension torque. Thus, FES-evoked torques were similar to the unparalyzed case, although targets were acquired about half as fast and with 4-times greater torque variability.

Despite greater torque variability overall, monkeys were able to grade FES-evoked torque using cell activity to acquire multiple targets in one direction. For example, monkey L produced 0.08±0.02 nM to acquire the near extension target and 0.14±0.02 nM to acquire the far extension target using the cell illustrated in FIG. 5. For all 5 cells tested, the torques produced to acquire graded torque targets in the same direction were significantly different (p<0.001). These cells acquired 11.3±5.0 graded torque targets/minute, comparable to the standard FES task (p=0.28), and made no target errors or extra attempts during peak performance.

During sessions in which monkey L simultaneously controlled two cell-muscle pairs to acquire bi-directional targets (5 pairs of cells), all performance measures were similar to single cell control (p>0.26). Both cells of each pair showed comparable performance measures. This monkey also improved with practice when using two cells simultaneously, acquiring 11.6±3.8 targets/minute during peak performance (compared to 3.8±1.4 targets/min at the beginning of practice; p=0.003; n=5), and requiring no additional attempts to reach each target (compared to extra attempts on 53% of targets at the beginning; p=0.008). Practice times with each cell pair ranged from 30 to 99 minutes (mean 56 min).

### Supplementary Discussion

Several factors may have limited tracking performance while the monkeys controlled FES via neural activity, including muscle fatigue, recording stability and training time. During some of the prolonged practice sessions muscle responses to electrical stimulation fatigued, as evidenced by a decrease in wrist torque evoked by a given stimulus intensity. This may have occurred because muscle stimulation preferentially activated large, fast-fatiguing motor units. Better methods to preserve a natural recruitment order of motor units and avoid fatigue could be employed in future studies, including stimulating over the motor point, via nerve cuffs, or in the spinal cord, which produces more natural recruitment order and fatigue-resistant contractions.

Given the numerous sensory projections to primary motor cortex, it was quite possible a priori that acute peripheral nerve block would disrupt volitional control of cells. Anesthetic doses sufficient to block both large and small diameter motor fibers also eliminate most sensory feedback from innervated areas. Nonetheless, 44 of 45 cells tested could be controlled after peripheral nerve block with similar or slightly greater modulation compared to the “unparalyzed” task. This is consistent with reports that tetraplegic patients with chronic spinal cord injury can modulate cell activity in motor cortex despite both a lack of sensory feedback, and the remodeling in cortical activity known to accompany chronic paralysis.

Our finding that cells could be used equally well to control FES, regardless of their original association to wrist movements, illustrates the flexibility of the cortical motor system. While strong correlations can be found between cortical activity and muscle force, for example, strong correlations can also be found with any number of movement parameters. Moreover, these correlations change, depending on the experimental conditions, and new correlations can emerge with brain control of cursors as well as FES.

The relatively direct control of FES from single neurons demonstrated here invites comparisons with the more conventional strategy of decoding movement-related activity from neural populations for prosthetic control. Given the rapid and robust operant control demonstrated for individual cells it seems remarkable that populations of cells used simultaneously have not provided more accurate control than they do. Several explanations are possible. First, demonstrating control of only one or two cells, with the rest of the brain free to optimize that control, is inherently simpler than controlling large numbers simultaneously. Second, prosthetic control functions defined on a population of neurons with stochastic variance may obscure the contribution of individual neurons and limit the brain's ability to learn to optimize those contributions. Third, the strategies that are typically used to optimize the transform from ensemble activity may undermine motor learning, by producing a ‘moving target’ through adaptive and frequently re-calibrated decoding methods. Instead, it may turn out that multiple direct connections from cortical cells to muscles will provide more explicit and intuitive functional connectivity that can be learned through innate motor learning mechanisms, particularly under conditions of long-term training.

An important next question for this approach is determining the degree to which multiple direct links from arbitrary cells can be used to control additional muscle patterns. Adding more channels could initially impose a greater cognitive load on the subject. Indeed, one rationale for the decoding approach is that tapping the natural activity patterns of appropriate cells would make control relatively intuitive. This decoding method assumes that the cells' correlation to behavior remains fixed; however most cells have volitionally controllable inputs that modulate their activity significantly to meet behavioral requirements. Consequently, the need to search for decodable cells may become less compelling if new but consistent sensorimotor contingencies can be learned with sufficient training time. The remarkable adaptability of cognitive motor control has been amply demonstrated, including the learning of BCI control: subjects that initially controlled a cursor through motor imagery soon began to imagine controlling the cursor directly. Maintaining long-term, stable recordings from isolated neurons is considered necessary for their use in neuroprosthetics, so achieving reliable isolation of the same units is a major goal of neural engineering. Using direct connections from single cells to control muscles would seem to be particularly vulnerable to loss of cell isolation. However, long-term isolation of the same units may not be a critical prerequisite, since humans and monkeys can rapidly learn to control external devices with newly acquired neural activity. In the present study monkeys improved control significantly despite an average of only 66 minutes of practice with each cell controlling FES. This would mean that obtaining new recorded cells or using more easily recorded multiunit activity could still provide effective control signals.

A clinically useful prosthetic control system of this type would probably employ multiple cells directly connected to many muscles. This would engage the inherent redundancy of the cortico-muscular system and help protect against loss of any single neuron. Single cells could also control functional muscle synergies, such as a coordinated contraction of many muscles to produce grasp. Given the likely development of improved implantable circuitry enabling long-term motor adaptation, the strategy of implementing relatively direct and separate connections from cortical cells to motor outputs offers a promising alternative to the current approach of intermingling signals from decoded neurons.

Throughout this application various publications, including patents and published patent applications, are referenced. The disclosures of these publications in their entireties are hereby incorporated by reference into this application in order to describe more fully the state of the art to which this invention pertains.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method of strengthening synaptic connections in a subject, the method comprising:
(a) detecting spike activity in a first neural site in the subject;
(b) delivering a stimulus pulse to a second neural site in the subject, wherein the stimulus pulse is delivered within 100 milliseconds of the detecting of a spike in step (a); and
(c) repeating steps (a) and (b) continuously for at least 12 hours;
whereby synaptic connections between the first and second neural sites are strengthened.

2. The method of claim 1, wherein the delivering of a stimulus pulse of step (b) is conditioned exclusively on the detecting of a spike in step (a) such that no stimulus pulse is delivered to the second neural site except for one stimulus pulse delivered within 50 milliseconds of each spike detected in the first neural site.

3. The method of claim 1, wherein the stimulation is intracortical microstimulation (ICMS).

4. The method of claim 3, wherein the pattern of neural activity in response to ICMS is determined by analyzing cortical activity.

5. The method of claim 1, wherein the first and second neural sites are in the cortex.

6. The method of claim 5, wherein the first and second neural sites are in the motor cortex.

7. The method of claim 6, wherein the pattern of neural activity in response to stimulation is determined by analyzing motor activity.

8. The method of claim 6, wherein the stimulus pulse delivered in step (b) is of an intensity below the movement threshold for a single pulse.

9. The method of claim 1, wherein the first and second neural sites are in the spinal cord.

10. The method of claim 1, wherein the first neural site is in the motor cortex and the second neural site is in the spinal cord.

11. The method of claim 1, wherein the detecting of step (a) comprises electromyography, and wherein the second neural site is in the spinal cord.

12. The method of claim 1, wherein the stimulus pulse delivered in step (b) has an intensity of about 10 μA-10 mA.

13. The method of claim 1, wherein steps (a) and (b) are repeated continuously for at least 24 hours.

14. The method of claim 1, wherein steps (a) and (b) are repeated continuously for at least 48 hours.

15. The method of claim 1, wherein the delivering of step (b) consists of delivering a stimulus pulse between 1 and 50 milliseconds after the detecting of step (a).

16. The method of claim 1, wherein the delivering of step (b) consists of delivering a stimulus pulse between 5 and 50 milliseconds after the detecting of step (a).

17. The method of claim 1, wherein the delivering of step (b) consists of delivering a stimulus pulse between 20 and 50 milliseconds after the detecting of step (a).

18. The method of claim 1, wherein the detecting of step (a) comprises obtaining a signal from the subdural or epidural recordings of the electrocorticogram (ECoG).

19. The method of claim 1, wherein the detecting of step (a) comprises obtaining a signal from single unit recording.

20. The method of claim 1, wherein the detecting of step (a) comprises obtaining a signal from the electromyogram (EMG).

21. The method of claim 1, wherein the stimulus of step (b) is delivered subdurally or epidurally.

22. The method of claim 1, wherein the conditioned neural response persists for at least one week.

23. A neural prosthesis comprising:
(a) means for detecting spike activity in a first neural site in a subject;
(b) means for delivering a stimulus pulse to a second site in the subject, wherein the stimulus pulse is delivered between 1 and 50 milliseconds after the detecting of step (a);
(c) means for conditioning delivery of a stimulus pulse to the second site exclusively on spike activity in the first neural site such that no stimulus pulse is delivered to the second site except for one stimulus pulse delivered within 50 milliseconds of each spike detected in the first neural site; and
(d) a support structure to which each of (a) through (c) is attached.

24. The device of claim 23, wherein the second site is a second neural site.

25. The device of claim 23, wherein the second site is a muscle.

26. The device of claim 23, wherein the support structure comprises a circuit board.

27. The device of claim 23, wherein the means for detecting spike activity comprises an electrode array.

28. The device of claim 23, wherein the means for delivering a stimulus pulse comprises an electrode array.

29. The device of claim 23, wherein the means for conditioning comprises a microprocessor or integrated circuit.
